(19) United States
(12) Patent Application Publication
(10) Pub. No.: US 2004/0175732 A1
(43) Pub. Date: Sep. 9, 2004

(54) Identification of micrornas and their targets

(76) Inventor: Tariq M. Rana, Shrewsbury, MA (US)

Correspondence Address: LAHIVE & COCKFIELD, LLP., 28 STATE STREET, BOSTON, MA, 02109, US

(21) Appl. No.: 10/715,231

(22) Filed: Nov. 17, 2003

Related U.S. Application Data
(60) Provisional application No. 60/426,912, filed on Nov. 15, 2002.
(60) Provisional application No. 60/458,059, filed on Mar. 26, 2003.

(51) Int. Cl. C12Q 1/68; C07H 21/04

(52) U.S. Cl. 435/6; 536/25.32

(57) ABSTRACT

The present invention relates to methods for identifying miRNAs and their targets in vivo and in vitro applicable to situations in which one or both of the sequences of the miRNA and the target nucleic acid is unknown. Further, the method applies to situations in which the miRNA/target interaction is stable or unstable; unstable reactions can be stabilized by the addition of crosslinking agents. The present invention also provides methods of modulating the expression of a target gene in a cell by modulating the activity of an miRNA in the cell that targets the gene.

### RELATED APPLICATIONS

This patent application claims the benefit of U.S. Provisional Patent Application Serial No. 60/426,912, entitled “Identification of microRNAS and Their Targets in vitro and in vivo”, filed Nov. 15, 2002 and U.S. Provisional Patent Application Serial No. 60/458,059, entitled “Identification of microRNAS and Their Targets”, filed Mar. 26, 2003. The entire contents of the above-referenced provisional patent applications are incorporated herein by this reference.

### GOVERNMENT FUNDING

This invention was made with Government grants from the National Institutes of Health (Nos. AI41404, AI45466, and AI43198). The U.S. Government has certain rights in this invention.

### BACKGROUND OF THE INVENTION

Developmentally important small noncoding RNAs, known as micro RNAs (miRNAs) have been identified in myriad organisms including nematodes, fruit flies, and humans. miRNAs are single-stranded, are generally 20-24 nucleotides (nt) in length, and are thought to be produced by processing of precursor molecules approximately seventy nucleotides in length that form a predicted RNA stem-loop structure. Many miRNAs appear to be evolutionarily conserved across species from worms to humans, and are believed to act by hybridizing with a target RNA. By a mechanism that is not completely understood, this interaction results in post-translational suppression of the target genes. Though the number of miRNAs described is steadily increasing, it is unknown whether each miRNA has only one target mRNA or multiple targets, and whether certain target mRNAs are regulated by more than one miRNA. The answers to these questions and others are vital to understanding the function and mechanism of miRNAs.

### SUMMARY OF THE INVENTION

The present invention relates to methods for identifying miRNAs and their targets in vivo and in vitro that can be applied both to situations in which one or both of the sequences of the miRNA and the target nucleic acid is unknown. Further, the method applies to situations in which the miRNA/target interaction is stable or unstable; unstable reactions can be stabilized by the addition of crosslinking agents. miRNA, either synthetic or natural, can be labeled with biotin and delivered to a cell or cells, or to cell extracts. In addition, psoralen-biotin and 4-thioU-biotin labeled miRNA can be added to cells and photocrosslinked to target RNA in vivo, thus identifying in vivo targets.

The present methods have several advantages. For instance, the sequences of miRNAs and their target RNAs can be determined without knowing the sequence of either beforehand, which allows for the identification of both novel miRNAs and their targets. The present methods are useful both in vitro and in vivo, allowing both the examination of gene regulation in vitro as well as in vivo, and the identification of gene regulation mechanisms that function in vivo. Where the sequence of an miRNA is known, the present invention provides methods for determining whether that miRNA has one target or multiple targets. Furthermore, the present invention provides for methods of stabilizing miRNA/target RNA interactions, so that even interactions which, while functional in vivo, would be insufficiently stable to detect using standard methods, may be detected using the present methods.

Other features and advantages of the invention will be apparent from the following detailed description, and from the claims.

### BRIEF DESCRIPTION OF THE DRAWINGS

FIG. 1A is a schematic diagram of one aspect of the present methods, as applied to identification of miRNAs and their targets when their interactions are stable; the sequence of the miRNA can be known or unknown.

FIG. 1B is a schematic diagram of one embodiment of the present methods, as applied to identification of miRNAs and their targets when their interactions are stable, and the sequence of the miRNA is known.

FIG. 1C is a schematic diagram of another embodiment, in which the target-complementary RNA (“tcRNA”) is extended from the 5′ end of the miRNA, before the adaptors are added.

FIG. 2A is a schematic diagram of one aspect of the present methods, as applied to identification of miRNAs and their targets when their interactions are dynamic; the sequence of the miRNA can be known or unknown.

FIG. 2B is a schematic diagram of another embodiment of the present methods, as applied to identification of miRNAs and their targets when their interactions are dynamic and the sequence of the miRNA is known.

FIG. 3 is a schematic diagram of one aspect of the present methods, as applied to identification of miRNAs and their targets when their interactions are stable, and the sequence of the miRNA is known.

FIG. 4 is a schematic diagram of another aspect of the present methods, as applied to identification of miRNAs and their targets when their interactions are dynamic, and the sequence of the miRNA is known.

FIG. 5A is an illustration of the structure of amino-modified cytosine.

FIG. 5B is an illustration of the structure of amino-modified uracil.

FIG. 6A is an illustration of the structure of Compound 1, bifunctional biotin-aminopentyl 8-hydroxypsoralen.

FIG. 6B is an illustration of the structure of Compound 2, an NHS activated ester of biotin butanoic acid.

FIG. 6C is an illustration of the structure of Compound 3, photocleavable biotin.

FIG. 7A is an illustration of the structure of Compound 4, an activated ester of hexanoic acid linked with a biotin and a 4-thio-uracil.

FIG. 7B is an illustration of the structure of Compound 5, an activated ester of hexanoic acid linked with a biotin and 8-hydroxy-psoralen.

FIG. 8A is a schematic diagram of another aspect of the present methods, which features a cDNA intermediate, as applied to the identification of miRNAs and their targets when their interactions are stable and the sequence of the miRNA is known.

FIG. 8B is a schematic diagram of another aspect of the present methods, which features a cDNA intermediate, as applied to the identification of miRNAs and their targets when their interactions are dynamic and the sequence of the miRNA is known.

FIG. 8C is a schematic diagram of one embodiment of the present methods, which features a cDNA intermediate and an miRNA having an amino-modified uridine or amino-modified cytosine, as applied to the identification of miRNAs and their targets when their interactions are dynamic and the sequence of the miRNA is known.

FIG. 8D is a schematic diagram of another embodiment of the present methods, which features a cDNA intermediate and an miRNA having a 4-thio uridine or thymidine or a 6-thio guanosine, as applied to the identification of miRNAs and their targets when their interactions are dynamic and the sequence of the miRNA is known.

FIG. 8E is a schematic diagram of another embodiment of the present methods, which features a cDNA intermediate and an miRNA having an amino-modified 3′ nucleotide, as applied to the identification of miRNAs and their targets when their interactions are dynamic and the sequence of the miRNA is known.

FIG. 8F is a schematic diagram of another embodiment of the present methods, which features a cDNA intermediate and an miRNA having an amino-modified 5′nucleotide, as applied to the identification of miRNAs and their targets when their interactions are dynamic and the sequence of the miRNA is known.

Like reference symbols in the various drawings indicate like elements.

### DETAILED DESCRIPTION OF THE INVENTION

MicroRNAs (miRNAs) are small, non-coding endogenous RNAs, putatively transcribed from larger (˜70 nt) stem-loop hairpin RNA structures that often contain a number of miRNAs. These small transcripts appear to function in an endogenous RNA interference mechanism of gene regulation, suppressing target genes to which they have some degree of complementarity (absolute complementarity is not required, and existing models only indicate 50-85% base pairing (McManus et al., RNA 8:842-850 (2002)). This mechanism of gene regulation is involved in normal development, and a number of the miRNAs elucidated thus far are evolutionarily conserved, appearing in species as diverse as nematodes and humans.

The present invention is based, in part, on the discovery of methods for elucidating the targets of miRNAs, whether or not the sequence of the miRNA is known. The methods of the present invention can be used both in vivo and in vitro. The methods are useful for determining the sequence of specific miRNAs and their targets, for investigating endogenous RNA interference-related mechanisms of regulating gene expression, for drug discovery, and for identifying therapeutic targets.

In one aspect, the invention features a method for identifying an miRNA and its target RNA, by first obtaining an miRNA/target RNA complex, either in vivo or in vitro. The complex can be crosslinked to increase stability if desired. Target complementary RNA (tcRNA) is transcribed from the target RNA; cDNA complementary to the tcRNA is synthesized, and the cDNA is then sequenced, either directly or after cloning.

The miRNA/target RNA complex may be obtained by obtaining miRNA, e.g,. by isolating total RNA and purifying the miRNA therefrom, administering the miRNA to a cell or cell extract; and allowing miRNA/target RNA complexes to form The miRNA/target RNA complex may be modified with a bifunctional biotin-aminopentyl 8-hydroxypsoralen (Compound 1) or any of the compounds described herein and photocrosslinked to increase stability. miRNA/target RNA complexes modified in this way can be immobilized using avidin-coated magnetic beads.

In another aspect, the invention features a method for identifying a target RNA of an miRNA by obtaining a modified miRNA comprising an amino-modified cytosine or amino-modified uracil; contacting the modified miRNA with a target RNA and allowing an miRNA/target RNA complex to form. The complex may be labeled with a biotin compound selected from an NHS activated ester of biotin butanoic acid (Compound 2) or photocleavable biotin (Compound 3), and target complementary RNA (tcRNA) can then be transcribed from the target RNA. Then, cDNA complementary to the tcRNA is synthesized and sequenced, either directly or after cloning. The modified miRNA/target RNA complex may be immobilized using avidin-coated magnetic beads.

In another aspect, the invention provides a method for identifying the target RNA of an miRNA by obtaining an miRNA having a known sequence, contacting the miRNA with a target RNA; allowing an miRNA/target RNA complex to form, and labeling the miRNA/target RNA complex with an activated ester of hexanoic acid linked with a biotin and a 4-thio-uracil (Compound 4) or an activated ester of hexanoic acid linked with a biotin and 8-hydroxy-psoralen (Compound 5). The miRNA/target RNA complex can be crosslinked to increase stability if desired, and target complementary RNA (tcRNA) can then be transcribed from the target RNA. Then, cDNA complementary to the tcRNA is synthesized and sequenced, either directly or after cloning. The modified miRNA/target RNA complex may be immobilized using avidin-coated magnetic beads.

In another aspect, the present invention features a method for identifying the target RNA of an miRNA, including the steps of: (a) contacting a biotin-labeled miRNA having a known sequence with a target RNA; (b) allowing an miRNA/target RNA complex to form; (c) crosslinking the miRNA/target RNA complex, e.g. with a psoralen compound or other crosslinking agent; immobilizing the complex on avidin-coated beads; reversing the crosslink; (d) transcribing a complementary strand from the target RNA using reverse transcriptase and a cDNA primer, e.g., a primer having a sequence corresponding to the miRNA; (e) synthesizing cDNA complementary to the transcribed strand; and (f) sequencing the cDNA, thereby identifying the target RNA.

In one embodiment, step (c) includes crosslinking the miRNA/target RNA complex via a modified nucleotide in the miRNA. In a preferred embodiment, the nucleotide is a uridine or cytidine within the miRNA. More preferably, the modified nucleotide is an amino-modified uridine or amino-modified cytidine within the miRNA.

In another preferred embodiment, the nucleotide is a uridine, thymidine or guanosine within the miRNA. More preferably, the modified nucleotide is a 4-thio uridine, 4-thio thymidine or 6-thio guanosine within the miRNA.

In another preferred embodiment, the crosslink is targeted to the 5′ end of the miRNA. Preferably, the crosslink comprises an amino modified 5′ nucleotide. More preferably, the crosslink comprises an amino-modified 5′ uridine or amino-modified 5′ cytidine.

In another preferred embodiment, the crosslink is targeted to the 3′ end of the miRNA. Preferably, the crosslink comprises an amino-modified 3′ nucleotide. More preferably, the crosslink comprises an amino-modified 3′ uridine or amino-modified 3′ cytidine.

In any of these methods, the miRNA/target RNA complex can form in a cell or in a cell-free solution.

In yet another aspect, the present invention provides a method for identifying a target RNA of an miRNA. This method includes the steps of: (a) contacting a cell with a psoralen-biotin conjugate such that the conjugate binds to target RNA within the cell; (b) allowing the target RNA to form a complex with miRNA within the cell; (c) immobilizing the miRNA:target RNA complex on avidin-coated beads; (d) reversing the crosslink; (e) transcribing a complementary strand from the target RNA using reverse transcriptase and a poly A primer; (f) synthesizing cDNA complementary to the transcribed strand of (f); and (g) sequencing the cDNA, thereby identifying the target RNA.

In still another aspect, the invention features a method for modulating the expression of a target RNA in a cell by identifying an miRNA that affects the expression of the target RNA using the present methods, and modulating the activity of the identified miRNA in the cell. The expression of the target RNA can be increased or decreased. In one embodiment, the target RNA encodes a gene involved in a proliferative or differentiative disease, such as the p53 gene, which is involved in cancer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

I. General Methodology

The present invention relies in part on the formation of stable miRNA/target RNA complexes; the stability of the complex can derive from either a high degree of complementarity between the miRNA and the target RNA, or from the use of crosslinking agents as described herein.

The starting materials are miRNAs and target RNAs. The miRNAs may be “natural,” e.g., isolated from cells, tissues, or organisms, or it may be synthetic, e.g., chemically synthesized or transcribed in vitro.

One significant benefit of the new methods is that the sequence of the miRNAs need not be known. The target RNA is generally obtained from cells, tissues, or organisms using routine methods; in some embodiments the target RNA may be in intact tissues or cells. Generally, the target RNA will be mRNA, but it may also be ribonucleoprotein complexes (RNPs) or other non-messenger RNA. In some embodiments, total RNA isolated from cells or cell extracts prepared by routine methods can be used as a source of target RNA. The formation of the miRNA/target RNA complex may take place in vitro, e.g., using isolated or synthetic miRNA and isolated target RNA, or may take place in vivo, e.g., using isolated or synthetic miRNA and target RNA contained in an intact cell.

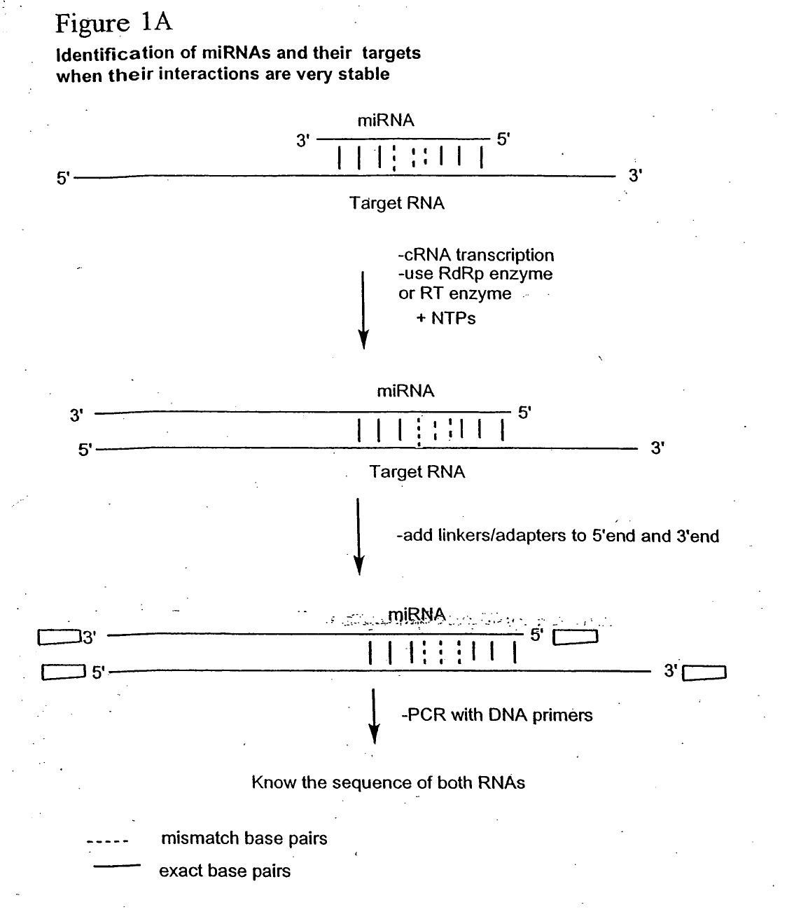

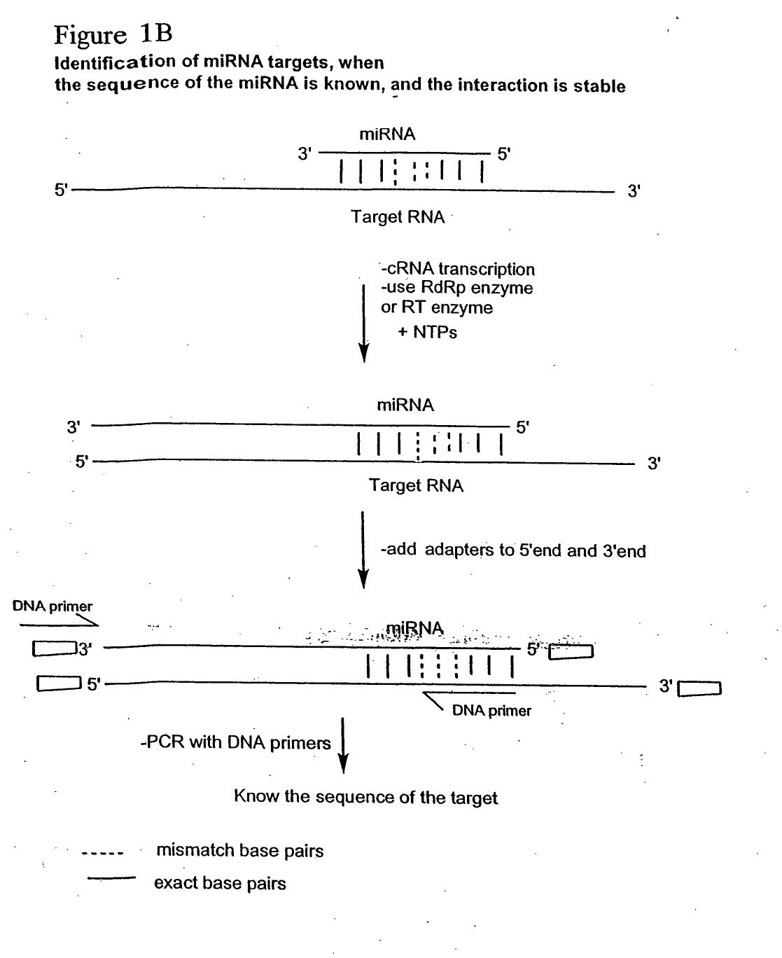

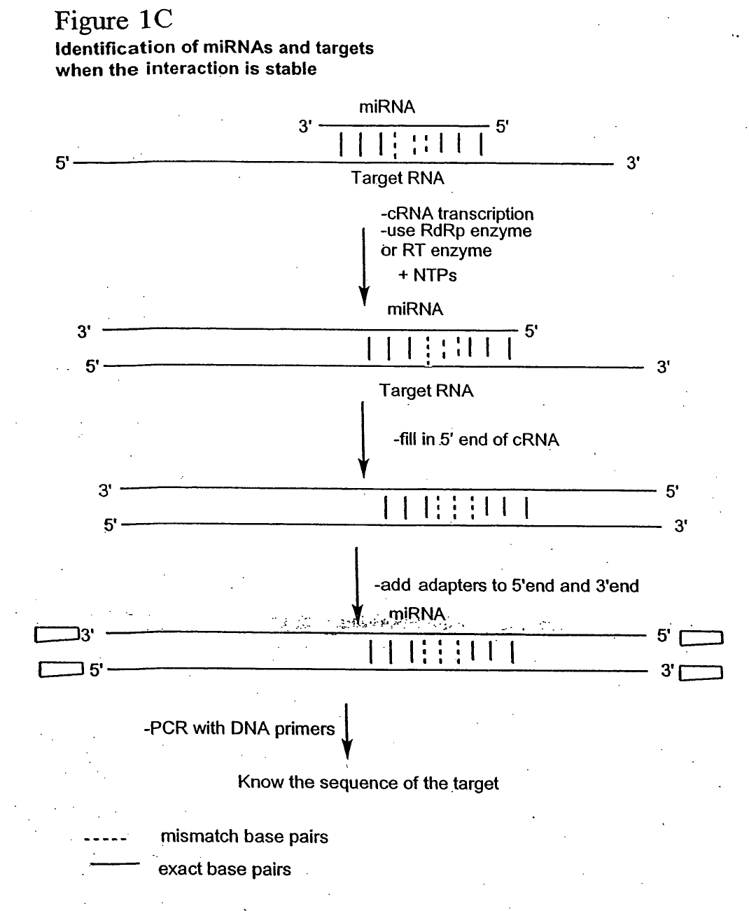

As stated previously, the sequence of the miRNA need not be known, as the present invention provides methods for isolating both the miRNA and its target RNA (or target RNAs, as there may be more than one target for each miRNA). One embodiment of the invention encompasses the situation where the sequence of the miRNA is not known but is isolated from cells or tissue using known techniques. As is illustrated in FIG. 1A, the miRNA is used as a primer to initiate the synthesis of a full-length ribonucleotide sequence complementary to the target RNA (this complementary sequence is referred to herein as “tcRNA”) by RNA dependent RNA polymerase (RdRP) or reverse transcriptase (RT) to form a double-stranded complex. Next, adapters of known sequences are ligated to one or more ends of the complex using DNA or RNA ligase enzymes and standard procedures, as discussed below. Then, using PCR primers complementary to the adapters, DNA can then be synthesized. The PCR products can be separated electrophoretically or on a column, and the sequences of RNAs can be deduced, either directly or after cloning. To clone the PCR products, standard methods can be used. For example, the ends of the miRNA/target RNA construct can be blunted and/or filled in using routine techniques, and the miRNA/target RNA complexes can be ligated into a blunt-ended cloning vector, cloned, and sequenced by routine methods. The PCR primers can be designed to have restriction enzyme recognition sequences to allow for convenient cloning.

Of course, this embodiment works equally well where the sequence of the miRNA is known or the miRNA is synthetic. Where the sequence of the miRNA is known, this embodiment can be modified such that one of the PCR primers can be designed to be homologous to the miRNA, as is shown in FIG. 1B.

FIG. 1C illustrates a further embodiment, in which the 5′ end of the miRNA is extended, towards the 3′ end of the target RNA. This is accomplished using standard methods, e.g., as described in Wang et al., Biochemistry 40:6458-6464 (2001).

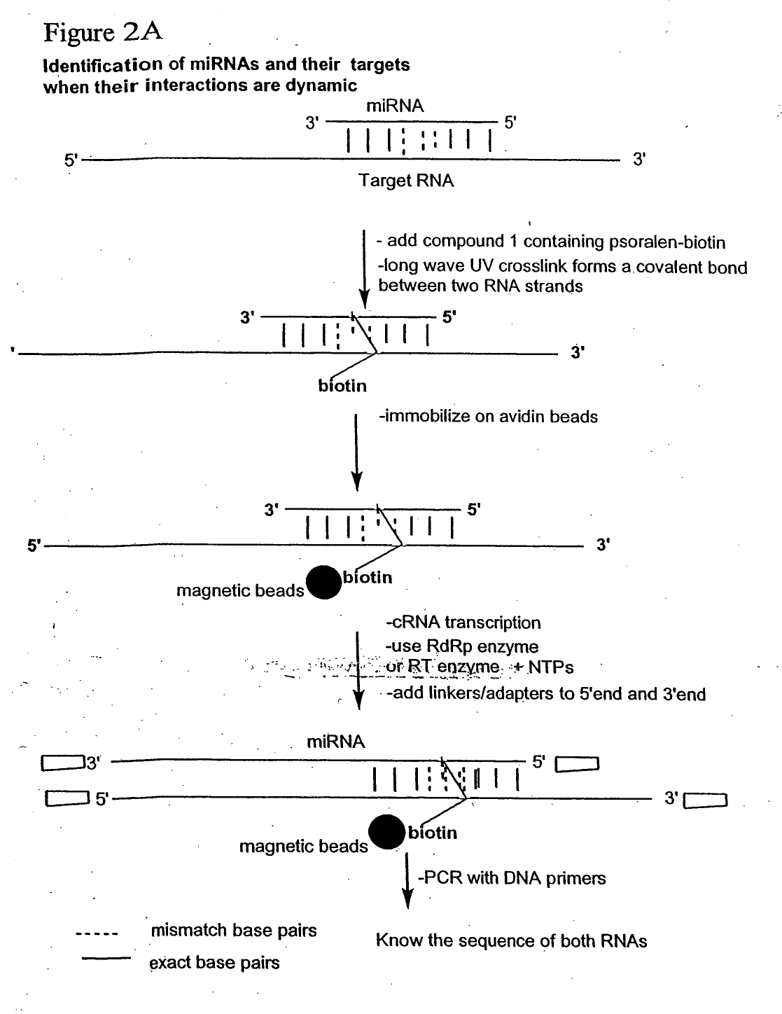

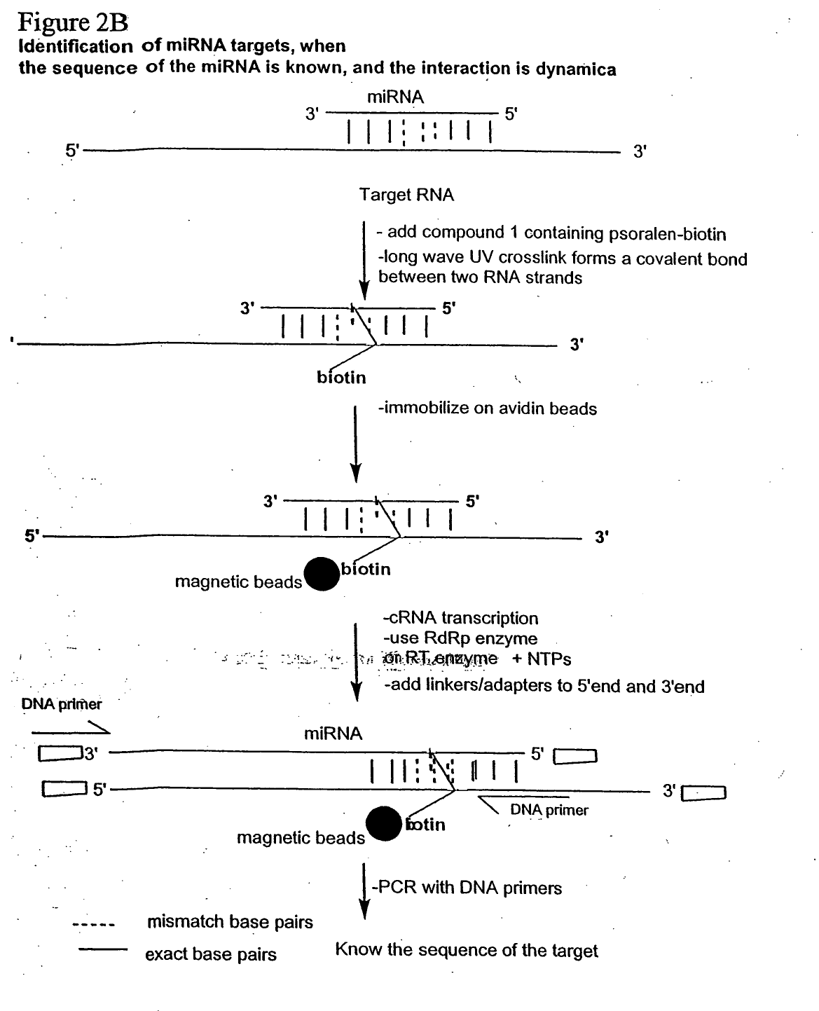

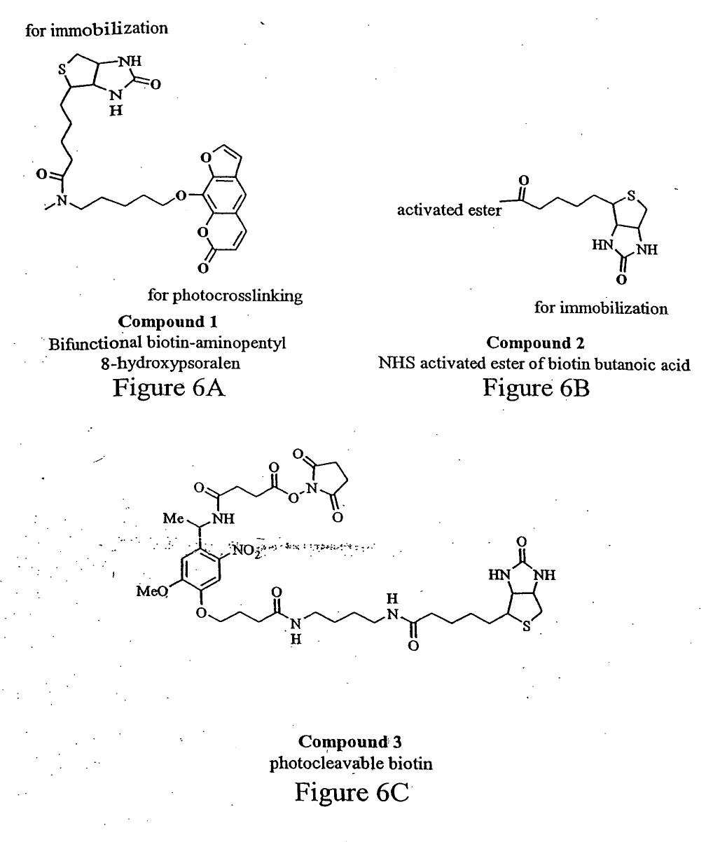

In certain situations, the interaction between the miRNA and the target RNA may be insufficiently stable for the miRNA to act as a primer. To overcome this instability (generally caused by lower overall complementarity between the miRNA and the target RNA, or a lack of complementarity at the ends of the miRNA), it is desirable to “freeze” or stabilize the dynamic interactions between miRNA and their targets. To achieve this, a bifunctional compound was synthesized with the formula shown for Compound 1, which is a bifunctional biotin-aminopentyl 8-hydroxypsoralen (see FIG. 6A). As is shown in FIGS. 2A and 2B, this compound can be added to the cells or cell extracts comprising miRNA/target RNA complexes, and exposed to long wave UV (360 nm) to freeze RNA-RNA interactions by crosslinking the strands. Alternatively, the compound can be added to the miRNA/target RNA complex formation mixture and then exposed to long wave UV (360 nm). Crosslinked miRNA/target RNA complexes can easily be immobilized on avidin-coated magnetic beads and further reactions can be carried out on beads or solution phase. As described above, RNA-dependent RNA polymerase (RdRP) or reverse transcriptase (RT) enzyme can be used to synthesize full-length tcRNA using the target RNA as a template, and the miRNA as a primer. Adapters of known sequences can be ligated to one or more of the ends of the tcRNA/target RNA complex using DNA or RNA ligase enzymes. Then, using PCR primers complementary to the adapters (FIG. 2A), DNA can be synthesized. The PCR primers can be designed to have restriction enzyme recognition sequences to allow for convenient cloning. The PCR products can be separated electrophoretically or on a column, and the sequences of RNAs can be deduced, either directly or after cloning. To clone the PCR products, standard methods can be used.

Again, this embodiment works equally well where the sequence of the miRNA is known or the miRNA is synthetic. Where the sequence of the miRNA is known, this embodiment can be modified so that one of the PCR primers can be designed to be homologous to the miRNA, as is shown in FIG. 2B. And although it is not shown, the 5′ end of the miRNA can be extended towards the 3′ end of the target RNA, using standard methods.

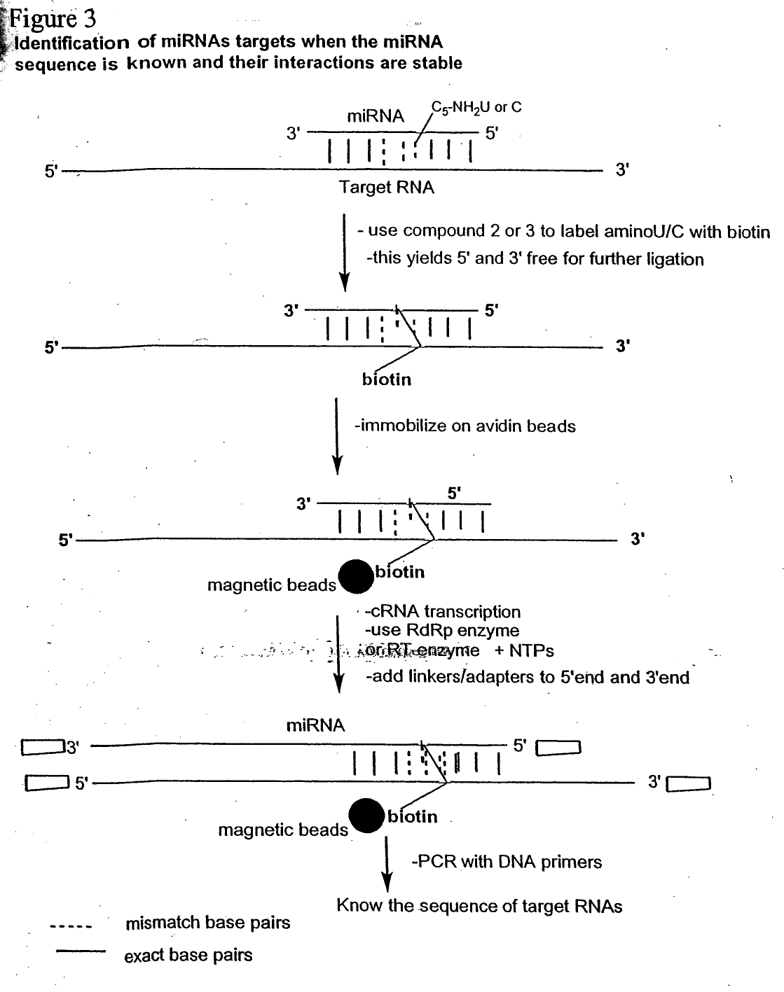

A large number of miRNA sequences are known but their targets are not known, and thus no function has been identified for them. Where the miRNA sequence is known, synthetic miRNAs can be made incorporating amino-modified C or U in the miRNA sequence. As shown in FIG. 3, further labeling of the modified bases with biotin or photocleavable biotin can be accomplished using Compound 2 (FIG. 6B) or Compound 3 (FIG. 6C). This labeled miRNA can be added to cells or cell extracts, and total RNA isolated; the miRNA/target RNA complex is then isolated using the biotin, e.g., using avidin-coated magnetic beads, and further reactions can be carried out on beads or solution phase. Again, as described above and shown in FIG. 3, RNA dependent RNA polymerase (RdRP) or reverse transcriptase (RT) enzyme can be used to synthesize full length tcRNA using the target RNA as a template and the miRNA as a primer. At this point, although it does not appear on FIG. 3, the 5′ end of the miRNA can be extended towards the 3′ end of the target RNA, using standard methods. Adapters of known sequences can be ligated to the ends of the tcRNA/target RNA complex using DNA or RNA ligase enzymes. Then, using PCR primers complementary to the adapters, DNA can be synthesized; in one embodiment one of the PCR primers can be designed to be homologous to the miRNA. The PCR primers can be designed to have restriction enzyme recognition sequences to allow for convenient cloning. The PCR products can be separated electrophoretically or on a column, and the sequences of RNAs can be deduced, either directly or after cloning. To clone the PCR products, standard methods can be used.

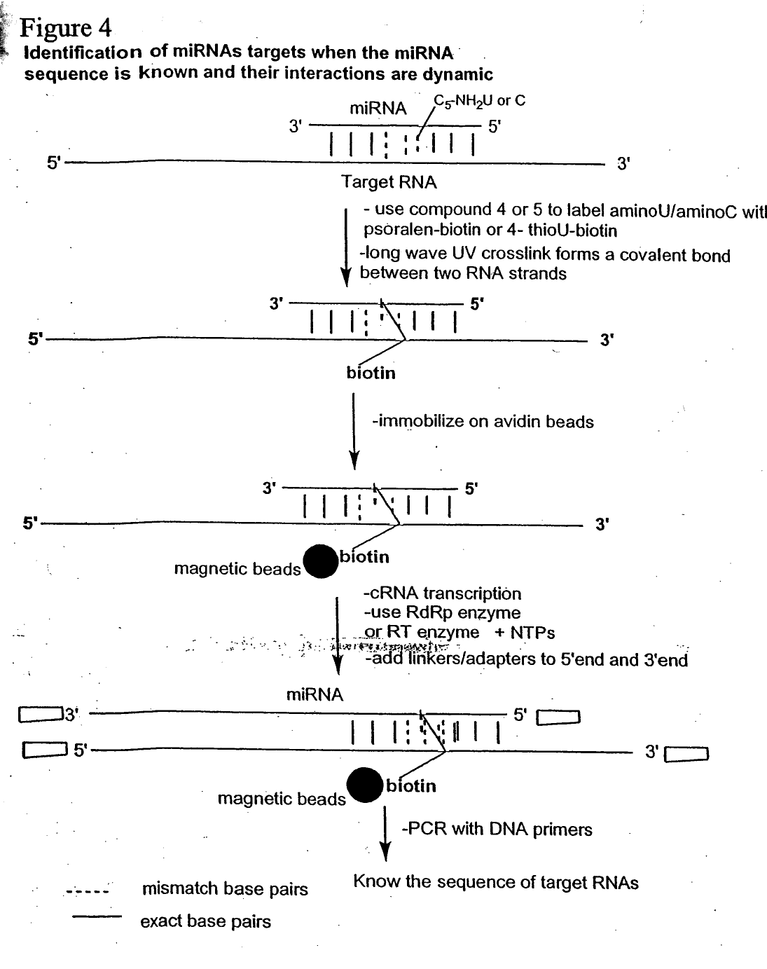

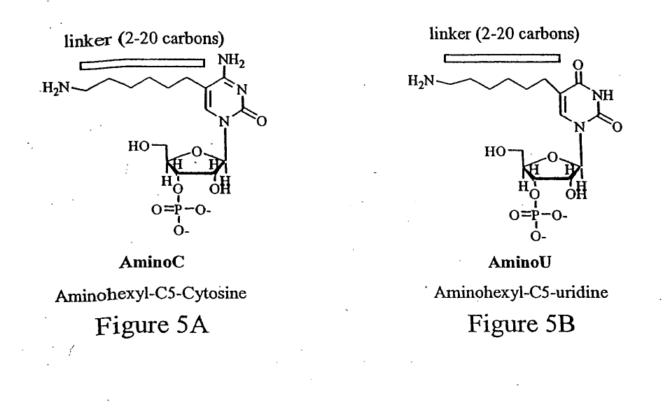

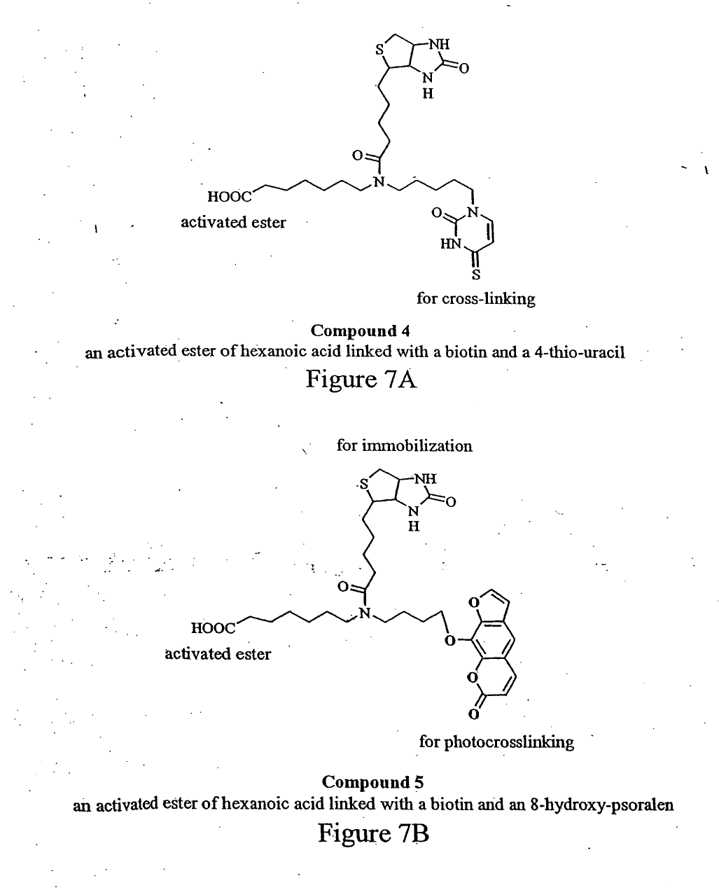

In the situation where sequence of the miRNA is known, and the interaction between the miRNA and the target RNA is dynamic, or insufficiently stable for the miRNA to act as a primer, the present invention provides a method for increasing stability and freezing dynamic interactions between miRNA and target RNA. As is shown in FIG. 4, Compound 4, a trifunctional activated ester of hexanoic acid linked with a biotin and a 4-thioUracil (FIG. 7A) or Compound 5, an activated ester of hexanoic acid linked with a biotin and an 8-hydroxy-psoralen (FIG. 7B) can be used to label the modified amino-U or amino-C in the synthetic miRNA. Either compound is added to the cells or cell extracts containing the modified miRNA, and then exposed to long wave UV (360 nm) that will freeze RNA-RNA interactions as described above. Alternatively, the compound can be added to the miRNA/target RNA complex formation mixture and then exposed to long wave UV (360 nm). Crosslinked miRNA/target RNA complexes can then be isolated by immobilization on magnetic beads and further reactions can be carried out on beads or solution phase. RNA dependent RNA polymerase (RdRP) or Reverse transcriptase (RT) enzyme can be used to synthesize full length tcRNA using target RNA as a template and the miRNA as a primer (as illustrated in FIG. 4). At this point, although it does not appear on FIG. 4, the 5′ end of the miRNA can be extended towards the 3′ end of the target RNA, using standard methods. Adapters of known sequences can be ligated to the ends of the tcRNA/target RNA complex by using DNA or RNA ligases. Then, using PCR primers complementary to the adapters (or, in some embodiments, homologous to the miRNA), DNA can be synthesized. The PCR primers can be designed to have restriction enzyme recognition sequences to allow for convenient cloning. The PCR products can be separated electrophoretically or on a column, and the sequences of RNAs can be deduced, either directly or after cloning. To clone the PCR products, standard methods can be used.

The above-described methods feature preparing the target RNA for amplification and eventual sequencing. In order to determine the sequence of the target, the miRNA that is bound to the target is used as a primer to initiate synthesis of a full length RNA complementary to the target RNA. The enzyme reverse transcriptase (RT) is used to generate the tcRNA which forms a full-length double-stranded complex with the target RNA. RT reads from the target, using the miRNA as a primer and extends the miRNA. However, it has been observed that the RT step is more efficient when priming comes from a DNA primer as compared to an RNA primer (i.e., an miRNA primer). Accordingly, additional strategies were designed in which the RT step involves a DNA primer.

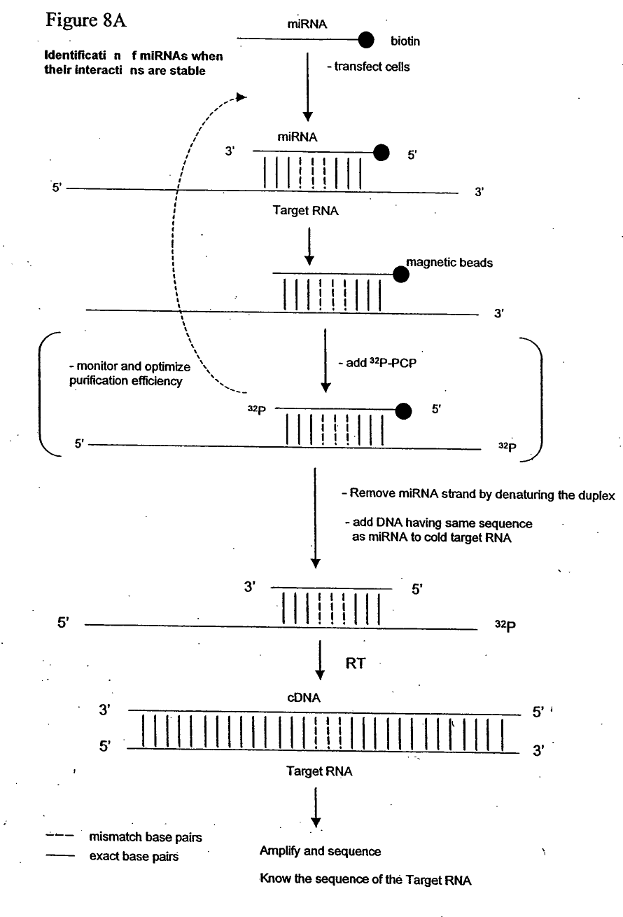

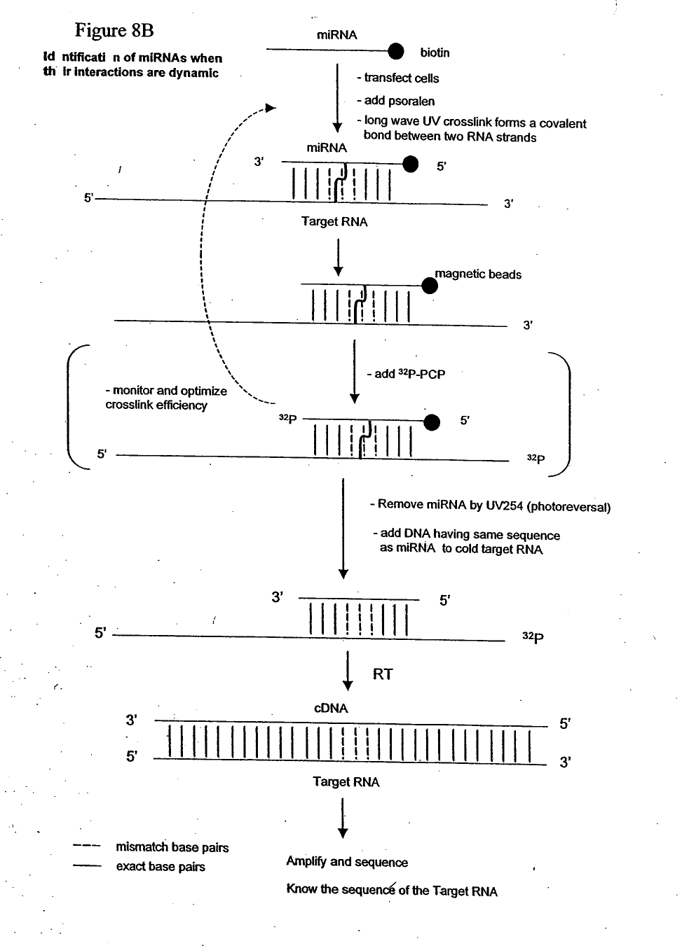

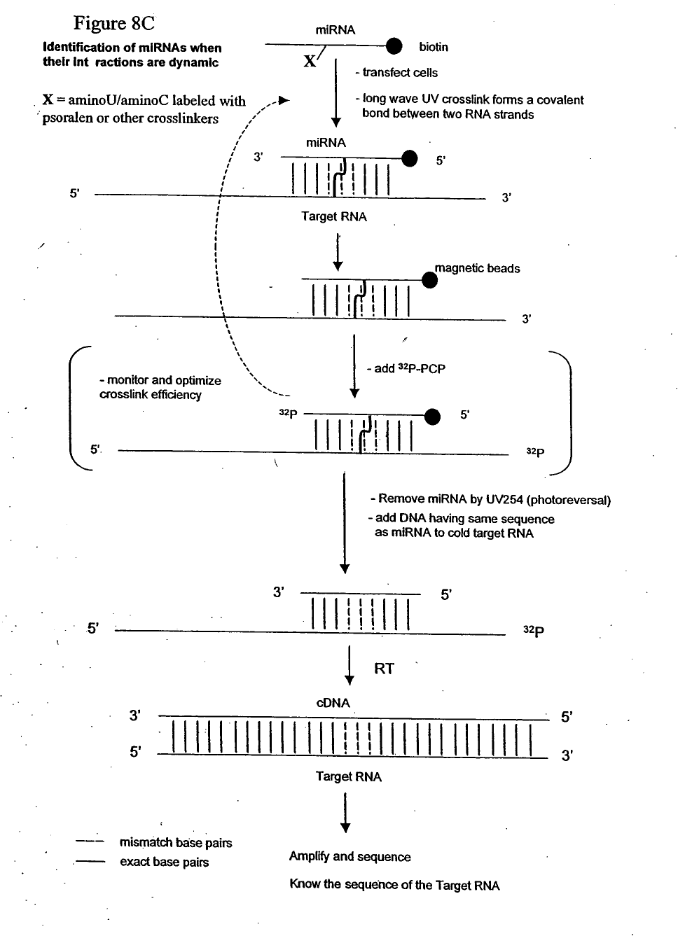

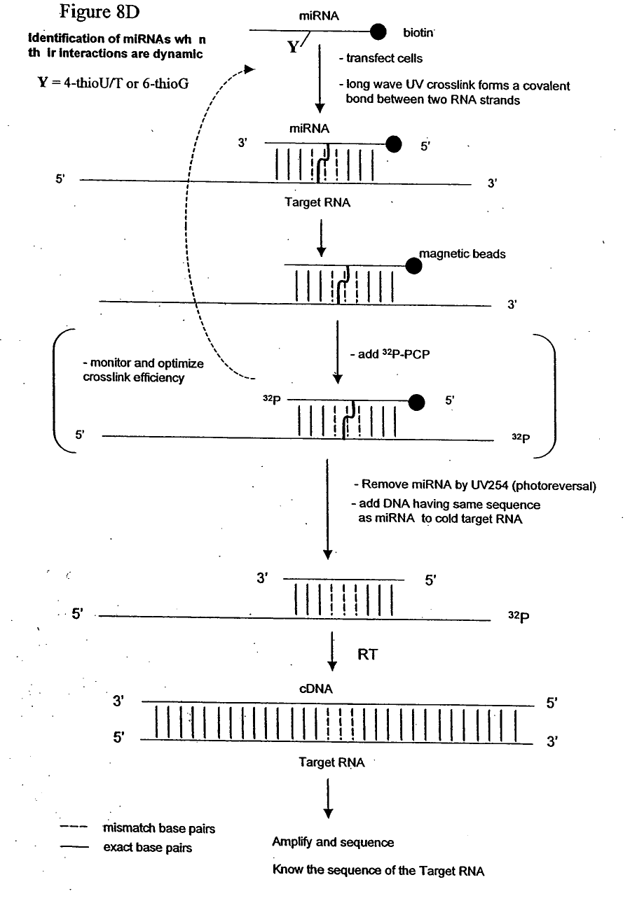

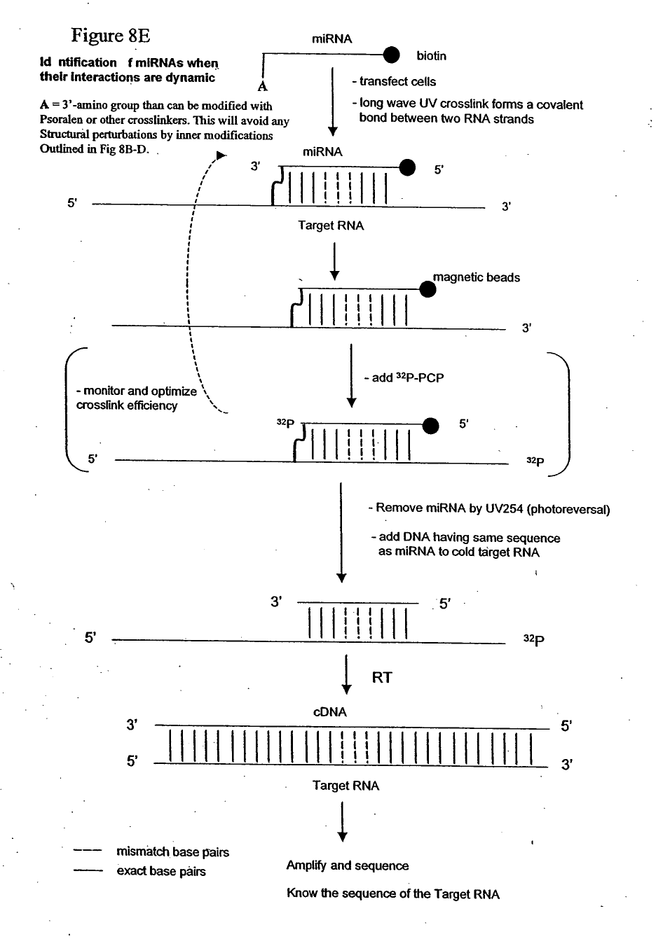

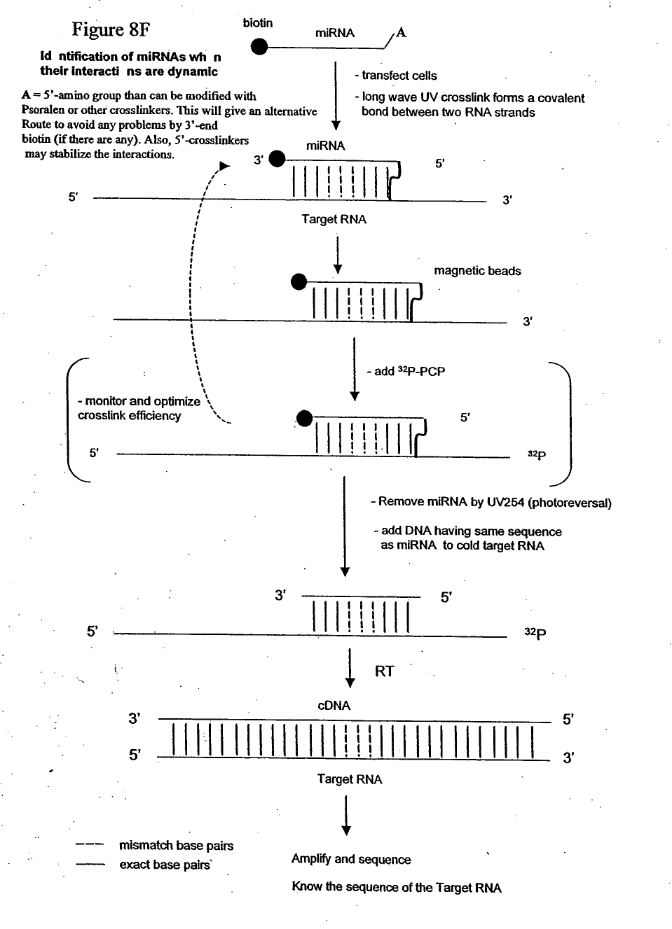

In one embodiment, the sequence of the miRNA is known. As illustrated in FIG. 8A, biotinylated miRNA is generated and transfected into a cell. miRNA-target RNA complexes are allowed to form. Total mRNA is isolated from the cell and streptavidin coated beads added to the extract. The beads bind to the biotin tag on the end of the miRNA and provide a convenient means to enrich for the miRNA-target complexes.

In an optional step, the enrichment can be monitored by using, PCP, e.g. α-³²P-cordycepin 5′ triphosphate, to label the 3′ end of the target mRNA, and radiolabeled RNA complexes can be separated by polyacrylamide gel electrophoresis. The detection of multiple radiolabeled bands, e.g. greater than 2, 3, 4 or 5 radiolabeled bands, can indicate the presence of contaminating RNA, e.g. non-target RNAs. The entire procedure can then be repeated and optimized for efficiency until one predominant band representing an miRNA-target complex is visible on the gel. Parameters that can be varied to optimize efficiency of the procedure include, but are not limited to, salt concentration, presence of non-denaturing detergents, e.g. NP-40 and sarkosyl, and the temperature at which the complex is formed.

The individual strands of the miRNA-target RNA duplex can be separated by heat denaturation, and the complementary DNA form of the miRNA can be added as a primer for subsequent synthesis (RT) reactions. The cDNA primer initiates the synthesis of a full-length sequence complementary to the target RNA by reverse transcriptase (RT) to form a double-stranded complex. The 5′ end of the cDNA primer can be extended towards the 3′ end of the target RNA using standard methods. Next, adapters of known sequences are ligated to one or more ends of the complex using DNA or RNA ligase enzymes and standard procedures. Then, using PCR primers complementary to the adapters, DNA can then be synthesized. An alternative to adding adapters and using adapter primers involves using random primers to initiate the PCR reaction. The PCR products can be separated electrophoretically or on a column, and the sequences of RNAs can be deduced, either directly or after cloning. To clone the PCR products, standard methods can be used. For example, the ends of the double-stranded complex can be blunted and/or filled in using routine techniques, and the complex ligated into a blunt-ended cloning vector, cloned, and sequenced by routine methods. The PCR primers can be designed to have restriction enzyme recognition sequences to allow for convenient cloning. In a preferred embodiment, one of the PCR primers can be designed to be homologous to the DNA primer used to prime the RT reaction.

In another embodiment in which the miRNA is known, the interaction between the miRNA and the target RNA is dynamic, or insufficiently stable for the miRNA to act as a primer. In this embodiment, as illustrated in FIG. 8B, biotinylated miRNA is generated and transfected into a cell, and miRNA-target RNA complexes are allowed to form, as described above. In order to increase complex stability, psoralen or a psoralen derivative and long wave UV light are used to cross-link the miRNA-target complex. Psoralens and psoralen derivatives that can be used in the present invention include, but are not limited to, 8-hydroxypsoralen, 8-((3-Idodopropyl-1)oxy)psoralen, aminomethyl psoralen, amino acid-modified psoralens, psoralen derivatives with modified stereochemistry, and psoralen derivatives with solubility in aqueous buffers. Total mRNA is isolated from the cell and streptavidin coated beads added to the extract to enrich for the miRNA target complexes.

In an optional step, the enrichment can be monitored by using PCP, e.g. α-³²P-cordycepin 5′ triphosphate, to label the 3′ end of the target mRNA, and radiolabeled RNA complexes can be separated by polyacrylamide gel electrophoresis. The detection of multiple radiolabeled bands, e.g. greater than 2, 3, 4 or 5 radiolabeled bands, can indicate the presence of contaminating RNA, e.g. non-target RNAs. The entire procedure can then be repeated and optimized for efficiency until one predominant band representing an miRNA-target complex is visible on the gel. Parameters that can be varied to optimize efficiency of the procedure include, but are not limited to, salt concentration, presence of non-denaturing detergents, e.g. NP-40 and sarkosyl, and the temperature at which the complex is formed.

Treating the isolated complex with photo-reversible UV 254 generates unmodified (i.e., uncross-linked) RNA species. The complementary DNA form of the miRNA can be added as a primer for subsequent synthesis (RT) reactions. The cDNA primer initiates the synthesis of a full-length sequence complementary to the target RNA by reverse transcriptase (RT) to form a double-stranded complex. The 5′ end of the cDNA primer can be extended towards the 3′ end of the target RNA using standard methods. Next, adapters of known sequences are ligated to one or more ends of the complex using DNA or RNA ligase enzymes and standard procedures. Then, using PCR primers complementary to the adapters, DNA can then be synthesized. An alternative to adding adapters and using adapter primers involves using random primers to initiate the PCR reaction. The PCR products can be separated electrophoretically or on a column, and the sequences of RNAs can be deduced, either directly or after cloning. To clone the PCR products, standard methods can be used. For example, the ends of the double-stranded complex can be blunted and/or filled in using routine techniques, and the complex ligated into a blunt-ended cloning vector, cloned, and sequenced by routine methods. The PCR primers can be designed to have restriction enzyme recognition sequences to allow for convenient cloning. In a preferred embodiment, one of the PCR primers can be designed to be homologous to the DNA primer sued to prime the RT reaction.

The above-described embodiment works equally well when the biotinylated miRNA is added to cell extracts or added to total RNA isolated from cells.

In another embodiment where the sequence is known and the interaction between the miRNA and the target RNA is dynamic, biotinylated miRNAs can be made incorporating amino-modified cytidine or amino-modified uridine in the miRNA sequence, as illustrated in FIG. 8C. The amino-modified bases are further modified with psoralen, or other crosslinkers known in the art. Other crosslinkers that can be used in the present invention include, but are not limited to, benzophenones, nitrogen mustards, and aryl-azides. The miRNA is transfected into a cell, and miRNA-target RNA complexes are allowed to form. Long wave UV light is used to cross-link the miRNA-target complex. Total mRNA is isolated from the cell and streptavidin coated beads added to the extract to enrich for the miRNA target complexes. Optionally, the efficiency of crosslinking of miRNA:target complexes can be monitored and optimized according to methods set forth above. Treating the isolated complex with photo-reversible UV 254 generates unmodified (i.e., uncross-linked) RNA species. The complementary DNA form of the miRNA can be added as a primer for subsequent synthesis (RT) reactions. The cDNA primer initiates the synthesis of a full-length sequence complementary to the target RNA by reverse transcriptase (RT) to form a double-stranded complex. The 5′ end of the cDNA primer can be extended towards the 3′ end of the target RNA using standard methods.

Next, adapters of known sequences are ligated to one or more ends of the complex using DNA or RNA ligase enzymes and standard procedures. Then, using PCR primers complementary to the adapters, DNA can then be synthesized. An alternative to adding adapters and using adapter primers involves using random primers to initiate the PCR reaction. The PCR products can be separated electrophoretically or on a column, and the sequences of RNAs can be deduced, either directly or after cloning. To clone the PCR products, standard methods can be used. For example, the ends of the double-stranded complex can be blunted and/or filled in using routine techniques, and the complex ligated into a blunt-ended cloning vector, cloned, and sequenced by routine methods. The PCR primers can be designed to have restriction enzyme recognition sequences to allow for convenient cloning. In a preferred embodiment, one of the PCR primers can be designed to be homologous to the DNA primer sued to prime the RT reaction.

The above-described embodiment works equally well when the biotinylated miRNA having amino-modifed cytidine or uridine is added to cell extracts or added to total RNA isolated from cells.

In another embodiment where the sequence is known and the interaction between the miRNA and the target RNA is dynamic, biotinylated miRNAs can be made incorporating 4-thio uridine or thymidine or 6-thio guanosine in the miRNA sequence, as illustrated in FIG. 8D. The 4-thio uridine, 4-thio thymidine or 6-thio guanosine can be incorporated into the RNA sequence of the miRNA according to methods known in the art, e.g. as described in Wang. et al. (1998) Biochemistry 37:4243, and Want et al. (1996) Biochemistry 35:6491-6499. The miRNA is transfected into a cell, and miRNA-target RNA complexes are allowed to form. Long wave UV light is used to cross-link the miRNA-target complex, e.g. 350-400 nm, as described in Wang. et al. (1998) Biochemistry 37:4243, and Want et al. (1996) Biochemistry 35:6491-6499. Total mRNA is isolated from the cell and streptavidin coated beads added to the extract to enrich for the miRNA target complexes. Optionally, the efficiency of crosslinking of miRNA:target complexes can be monitored and optimized according to methods set forth above. Treating the isolated complex with photo-reversible UV 254 generates unmodified (i.e., uncross-linked) RNA species. The complementary DNA form of the miRNA can be added as a primer for subsequent synthesis (RT) reactions. The cDNA primer initiates the synthesis of a full-length sequence complementary to the target RNA by reverse transcriptase (RT) to form a double-stranded complex. The 5′ end of the cDNA primer can be extended towards the 3′ end of the target RNA using standard methods.

Next, adapters of known sequences are ligated to one or more ends of the complex using DNA or RNA ligase enzymes and standard procedures. Then, using PCR primers complementary to the adapters, DNA can then be synthesized. An alternative to adding adapters and using adapter primers involves using random primers to initiate the PCR reaction. The PCR products can be separated electrophoretically or on a column, and the sequences of RNAs can be deduced, either directly or after cloning. To clone the PCR products, standard methods can be used. For example, the ends of the double-stranded complex can be blunted and/or filled in using routine techniques, and the complex ligated into a blunt-ended cloning vector, cloned, and sequenced by routine methods. The PCR primers can be designed to have restriction enzyme recognition sequences to allow for convenient cloning. In a preferred embodiment, one of the PCR primers can be designed to be homologous to the DNA primer sued to prime the RT reaction.

The above-described embodiment works equally well when the biotinylated miRNA having 4-thio urdine or thymidine or 6-thio guanosine is added to cell extracts or added to total RNA isolated from cells.

The above-described strategies, in which the RT step involves a DNA primer, feature miRNA sequences having internal modified bases. Further labeling of the internal modified bases with psoralen or other crosslinkers and photo-activation facilitates crosslinking at the internal bases with the target RNA. However, internal modifications could affect the interaction between the miRNA and target RNA. Accordingly, the instant invention provides additional strategies for crosslinking the miRNA target complex at the 5′ or 3′ end of the miRNA.

In one embodiment where the sequence is known and the interaction between the miRNA and the target RNA is dynamic, biotinylated miRNAs can be generated having an amino-modified nucleotide at the 3′ end, as illustrated in FIG. 8E. The amino-modified nucleotide can be nucleobase-modified, e.g. amino-modified uridine or amino-modified cytosine, or sugar-modified, e.g. by modification at the 3′ position of the sugar with an amino-ethylene group with carbon linkers, e.g. 3-12 carbon linkers. The amino-modified 3′ nucleotide is further modified with psoralen, or other crosslinkers known in the art, as set forth above. Modification at the 3′ end may avoid potential structural perturbations induced by internal modifications set forth above. The miRNA is transfected into a cell, and miRNA-target RNA complexes are allowed to form. Long wave UV light is used to cross-link the miRNA-target complex. Total mRNA is isolated from the cell and streptavidin coated beads added to the extract to enrich for the miRNA target complexes. Optionally, the efficiency of crosslinking of miRNA:target complexes can be monitored and optimized according to methods set forth above. Treating the isolated complex with photo-reversible UV 254 generates unmodified (i.e., uncross-linked) RNA species. The complementary DNA form of the miRNA can be added as a primer for subsequent synthesis (RT) reactions. The cDNA primer initiates the synthesis of a full-length sequence complementary to the target RNA by reverse transcriptase (RT) to form a double-stranded complex. The 5′ end of the cDNA primer can be extended towards the 3′ end of the target RNA using standard methods.

Next, adapters of known sequences are ligated to one or more ends of the complex using DNA or RNA ligase enzymes and standard procedures. Then, using PCR primers complementary to the adapters, DNA can then be synthesized. An alternative to adding adapters and using adapter primers involves using random primers to initiate the PCR reaction. The PCR products can be separated electrophoretically or on a column and the sequences of RNAs can be deduced, either directly or after cloning. To clone the PCR products, standard methods can be used. For example, the ends of the double-stranded complex can be blunted and/or filled in using routine techniques, and the complex ligated into a blunt-ended cloning vector, cloned, and sequenced by routine methods. The PCR primers can be designed to have restriction enzyme recognition sequences to allow for convenient cloning. In a preferred embodiment, one of the PCR primers can be designed to be homologous to the DNA primer used to prime the RT reaction.

The above-described embodiment works equally well when the biotinylated miRNA having an amino-modified nucleotide at the 3′ end is added to cell extracts or added to total RNA isolated from cells.

In another embodiment, biotinylated miRNAs can be generated having an amino-modified nucleotide at the 5′ end, as illustrated in FIG. 8F. The amino-modified nucleotide can be nucleobase-modified, e.g. amino-modified uridine or amino-modified cytosine, or sugar-modified, e.g. by modification at the 5′ position of the sugar with an amino-ethylene group with carbon linkers, e.g. 3-12 carbon linkers. The amino-modified nucleotide at the 5′ end is further modified with psoralen, or other crosslinkers known in the art, as set forth above. Modification at the 5′ end may avoid potential structural perturbations induced by internal modification and potential problems caused by modification at the 3′ end. Modification at the 5′ end may also stabilize the interaction between the miRNA and target RNA. The miRNA is transfected into a cell, and miRNA-target RNA complexes are allowed to form. Long wave UV light is used to cross-link the miRNA-target complex. Total mRNA is isolated from the cell and streptavidin coated beads added to the extract to enrich for the miRNA target complexes. Optionally, the efficiency of crosslinking of miRNA:target complexes can be monitored and optimized according to methods set forth above. Treating the isolated complex with photo-reversible UV 254 generates unmodified (i.e., uncross-linked) RNA species. The complementary DNA form of the miRNA can be added as a primer for subsequent synthesis (RT) reactions. The cDNA primer initiates the synthesis of a full-length sequence complementary to the target RNA by reverse transcriptase (RT) to form a double-stranded complex. The 5′ end of the cDNA primer can be extended towards the 3′ end of the target RNA using standard methods.

Next, adapters of known sequences are ligated to one or more ends of the complex using DNA or RNA ligase enzymes and standard procedures. Then, using PCR primers complementary to the adapters, DNA can then be synthesized. An alternative to adding adapters and using adapter primers involves using random primers to initiate the PCR reaction. The PCR products can be separated electrophoretically or on a column, and the sequences of RNAs can be deduced, either directly or after cloning. To clone the PCR products, standard methods can be used. For example, the ends of the double-stranded complex can be blunted and/or filled in using routine techniques, and the complex ligated into a blunt-ended cloning vector, cloned, and sequenced by routine methods. The PCR primers can be designed to have restriction enzyme recognition sequences to allow for convenient cloning. In a preferred embodiment, one of the PCR primers can be designed to be homologous to the DNA primer used to prime the RT reaction.

The above-described embodiment works equally well when the biotinylated miRNA having an amino-modified nucleotide at the 5′ end is added to cell extracts or added to total RNA isolated from cells.

In another embodiment, the sequence of the miRNA is not known. In this embodiment, a psoralen-biotin conjugate with a linker (e.g., Compound 1, FIG. 6A) is added to cells and binds to target RNA within the cells. Crosslinked products are isolated using streptavidin beads and the protocol proceeds essentially as described above. However, as the miRNA sequence is not known, a poly-A primer is used in place of the cDNA primer to initiate synthesis of the full-length sequence complementary to the target RNA. Reverse transcriptase (RT) is used to form the double-stranded complex. Again, adapters of known sequences are ligated to one or more ends of the complex using DNA or RNA ligase enzymes and standard procedures. An adapter primer is used for the PCR step needed to amplify the target prior to sequencing. Alternatively, random primers can be used, as described above.

In some embodiments, the miRNA may bind to a target that is other than an RNA, e.g., protein or protein complexes, e.g., RNA-binding proteins, RNA-DNA hybrids, and the like. In this case, the miRNA/non-RNA-target complex can be separated using SDS-PAGE, and the protein bands can be isolated and sequenced by MALDI MS mass spectrometry techniques routine in the art.

II. Methods of Treatment

The present invention provides methods for identifying miRNAs and their targets in vitro and in vivo, which is useful clinically (e.g., in certain prophylactic and/or therapeutic applications). For example, miRNAs can be used, for example, as prophylactic and/or therapeutic agents in the treatment of diseases or disorders associated with unwanted or aberrant expression of the corresponding target gene.

In one embodiment, the invention provides for prophylactic methods of treating a subject at risk of (or susceptible to) a disease or disorder, for example, a disease or disorder associated with aberrant or unwanted target gene expression or activity. Subjects at risk for a disease which is caused or contributed to by aberrant or unwanted target gene expression or activity can be identified by, for example, any or a combination of diagnostic or prognostic assays as described herein. Administration of a prophylactic agent can occur prior to the manifestation of symptoms characteristic of the target gene aberrancy, such that a disease or disorder is prevented or, alternatively, delayed in its progression. Depending on the type of target gene aberrancy, for example, a target gene, target gene agonist or target gene antagonist agent can be used for treating the subject. The appropriate agent can be determined based on screening assays described herein.

In another embodiment, the invention provides for therapeutic methods of treating a subject having a disease or disorder, for example, a disease or disorder associated with aberrant or unwanted target gene expression or activity. In an exemplary embodiment, the modulatory method of the invention involves contacting a cell capable of expressing target gene with a therapeutic agent that is specific for the target gene or protein (e.g., is specific for the mRNA encoded by said gene or specifying the amino acid sequence of said protein) such that expression or one or more of the activities of target protein is modulated. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant or unwanted expression or activity of a target gene polypeptide or nucleic acid molecule. Inhibition of target gene activity is desirable in situations in which target gene is abnormally unregulated and/or in which decreased target gene activity is likely to have a beneficial effect.

“Treatment”, or “treating” as used herein, is defined as the application or administration of a prophylactic or therapeutic agent to a patient, or application or administration of a prophylactic or therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

A preferred aspect of the invention features a method for modulating the expression of a target RNA in a cell by identifying an miRNA that affects the expression of the target RNA using the present methods, and modulating the activity of the identified miRNA in the cell. The expression of the target RNA can be increased or decreased. In one embodiment, the target RNA encodes a gene involved in a proliferative or differentiative disease or disorder.

As used herein, the term “proliferative disease” or “proliferative disorder” refers to a condition in which abnormal or unwanted cell proliferation occurs in of one or more cells or populations of cells in a mammal, such as a human, resulting in an abnormal state or condition in the mammal. As used herein, the term “differentiative disease” or “differentiative disorder” refers to a condition in which abnormal or altered cell differentiation occurs in of one or more cells or populations of cells in a mammal, such as a human, resulting in an abnormal state or condition in the mammal.

Typically, a “proliferative disease” or “disorder” is caused by cells (e.g., somatic cells) which grow more quickly than normal cells, leading to an abnormal mass of tissue, the growth of which exceeds and is uncoordinated with that of the normal tissues.

Often abnormal cell proliferation is coupled with altered cell differentiative capacity, in particular, decreased differentiative capacity or dedifferentiation.

Examples of proliferative and/or differentiative diseases or disorders include cancer, e.g., carcinomas, sarcomas, metastatic disorders or hematopoietic neoplastic disorders, e.g., leukemias, as well as proliferative skin disorders, e.g., psoriasis or hyperkeratosis. Other myeloproliferative disorders include polycythemia vera, myelofibrosis, chronic myelogenous (myelocytic) leukemia, and primary thrombocythaemia, as well as acute leukemia, especially erythroleukemia, and paroxysmal nocturnal haemoglobinuria. Metastatic tumors can arise from a multitude of primary tumor types, including but not limited to those of prostate, colon, lung, breast and liver origin.

As used herein, the terms “cancer,” “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. “Benign hyperproliferative” cells can include non-malignant tumor cells, such as are associated with benign prostatic hyperplasias, hepatocellular adenomas, hemangiomas, focal nodular hyperplasias, angiomas, dysplastic nevi, lipomas, pyogenic granulomas, seborrheic keratoses, dermatofibromas, keratoacanthomas, keloids, and the like.

The terms “cancer” or “neoplasms” include malignancies of the various organ systems, such as affecting lung, breast, thyroid, lymphoid, gastrointestinal, and genitourinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L., Ball, E. D., Foon, K. A. (1991) Immune markers in hematologic malignancies. Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Examples of genes involved in proliferative and/or differentiative disorders include but are not limited to oncogenes, e.g., genes associated with stimulation of cell division, including growth factors or receptors for growth factors, e.g., PDGF, PDGF-R, RET, erb-B, erb-B2; cytoplasmic signalling molecules, e.g., Ki-ras, N-ras, and c-src; transcription factors, e.g., c-myc, N-myc, L-myc, c-jun, and c-fos; and others, e.g., Bcl-2, Bcl-1/cyclin D1, and MDM2; and tumor suppressor genes, e.g., genes associated with inhibition of cell division, e.g., cytoplasmic proteins, e.g., APC, DPC4, NF-1, and NF-2; nuclear proteins, e.g., MTS1, RB/pRB, p53, p16, WT1, BRCA1, and BRCA2; and others, e.g., VHL.

In an exemplary embodiment, an miRNA identified by the new methods to target mRNA for p53, a tumor suppressor protein, can be used to modulate the function of the p53 mRNA. Increasing the levels of miRNAs specific for p53 either by increasing levels of the p53 miRNA precursor gene, e.g., by increasing transcription or decreasing degradation, or by addition of exogenous synthetic or natural miRNA, would result in decreased translation of the p53 gene, which would be useful in both creating and testing animal and cellular models of oncogenic processes, as well as in cancer therapy.

In another aspect of the invention, methods that interfere with the functioning of miRNA regulation of genes can be used to increase gene translation, such as the use of antisense or RNAi techniques to decrease expression of the miRNA precursor gene, the miRNA itself, or other genes that are required for the proper regulation of genes by miRNA. One such target would be a mammalian factor involved in the miRNA-mediated regulation of genes, similar to RdRP in the worm; thus, increased or decreased expression of an RdRP-type factor may enhance or decrease miRNA regulation (see, e.g., Lipardi et al., Cell 107(3):297-307 (2001)).

Further, since miRNA is believed to be involved in translational control, knowledge of miRNAs and their targets would allow specific modulation of miRNA systems to treat any of a number of disorders (including cancer, inflammation, neuronal disorders, etc.) controlled at the translational level. Manipulating miRNA regulation of translation of these genes is a novel, powerful, and specific method for treating these disorders.

III. Pharmacopenomics and Pharmaceutical Compositions

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment with either the target gene molecules of the present invention or target gene modulators according to that individual's drug response genotype. Pharmacogenomics allows a clinician or physician to target prophylactic or therapeutic treatments to patients who will most benefit from the treatment and to avoid treatment of patients who will experience toxic drug-related side effects.

With regards to the above-described agents for prophylactic and/or therapeutic treatments (e.g., miRNAs or miRNA precursors), the agents are routinely incorporated into pharmaceutical compositions suitable for administration. Such compositions typically comprise the nucleic acid molecule, protein, antibody, or modulatory compound and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, (Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. Although compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the EC50 (i.e., the concentration of the test compound which achieves a half-maximal response) as determined in cell culture. Such information can be used to more accurately determine useful doses in humans. Levels in plasma may be measured, for example, by high performance liquid chromatography.

When administering miRNAs (e.g., naturally occurring miRNAs or synthetic miRNAs), it may be advantageous to chemically modify the miRNA in order to increase in vivo stability. Preferred modifications stabilize the miRNA against degradation by cellular nucleases.

The pharmaceutical compositions can be included in a container, pack, or dispenser together with instructions for administration.

IV. Experimental Applications

As discussed supra., the present invention provides methods for identifying miRNAs and their targets, both in vitro and in vivo. miRNAs and their targets so identified can further be used experimentally, for example, in creating knockout and/or knockdown cells or organisms, in functional genomics and/or proteomics applications, in screening assays, and the like.

A. Knockout and/or Knockdown Cells or Organisms

A miRNAs (either known or identified by the methodologies of the present invention) can be used in a functional analysis of the corresponding target RNA (either known or identified by the methodologies of the present invention). Such a functional analysis is typically carried out in eukaryotic cells, or eukaryotic non-human organisms, preferably mammalian cells or organisms and most preferably human cells, e.g. cell lines such as HeLa or 293 or rodents, e.g. rats and mice. By administering a suitable miRNA molecule, a specific knockout or knockdown phenotype can be obtained in a target cell, e.g. in cell culture or in a target organism.

Thus, further subject matter of the invention includes cells (e.g., eukaryotic cells) or organisms (e.g., eukaryotic non-human organisms) exhibiting a target gene-specific knockout or knockdown phenotype resulting from a fully or at least partially deficient expression of at least one endogeneous target gene wherein said cell or organism is transfected with or administered, respectivey, at least one miRNA, vector comprising DNA encoding said miRNA (or an miRNA precursor) capable of inhibiting the expression of the target gene. It should be noted that the present invention allows a target-specific knockout or knockdown of several different endogeneous genes based on the specificity of the miRNAi(s) transfected or administered.

Gene-specific knockout or knockdown phenotypes of cells or non-human organisms, particularly of human cells or non-human mammals may be used in analytic to procedures, e.g. in the functional and/or phenotypical analysis of complex physiological processes such as analysis of gene expression profiles and/or proteomes. Preferably the analysis is carried out by high throughput methods using oligonucleotide based chips.

Using RNAi based knockout or knockdown technologies, the expression of an endogeneous target gene may be inhibited in a target cell or a target organism. The endogeneous gene may be complemented by an exogenous target nucleic acid coding for the target protein or a variant or mutated form of the target protein, e.g. a gene or a DNA, which may optionally be fused to a further nucleic acid sequence encoding a detectable peptide or polypeptide, e.g an affinity tag, particularly a multiple affinity tag.

Variants or mutated forms of the target gene differ from the endogeneous target gene in that they encode a gene product which differs from the endogeneous gene product on the amino acid level by substitutions, insertions and/or deletions of single or multiple amino acids. The variants or mutated forms may have the same biological activity as the endogeneous target gene. On the other hand, the variant or mutated target gene may also have a biological activity, which differs from the biological activity of the endogeneous target gene, e.g. a partially deleted activity, a completely deleted activity, an enhanced activity etc. The complementation may be accomplished by compressing the polypeptide encoded by the endogeneous nucleic acid, e.g. a fusion protein comprising the target protein and the affinity tag and the double stranded RNA molecule for knocking out the endogeneous gene in the target cell. This compression may be accomplished by using a suitable expression vector expressing both the polypeptide encoded by the endogenous nucleic acid, e.g. the tag-modified target protein and the double stranded RNA molecule or alternatively by using a combination of expression vectors. Proteins and protein complexes which are synthesized de novo in the target cell will contain the exogenous gene product, e.g., the modified fusion protein. In order to avoid suppression of the exogenous gene product by the siRNAi molecule, the nucleotide sequence encoding the exogenous nucleic acid may be altered at the DNA level (with or without causing mutations on the amino acid level) in the part of the sequence which so is homologous to the siRNA molecule. Alternatively, the endogeneous target gene may be complemented by corresponding nucleotide sequences from other species, e.g. from mouse.

B. Functional Genomics and/or Proteomics

Preferred applications for the cell or organism of the invention is the analysis of gene expression profiles and/or proteomes. In an especially preferred embodiment an analysis of a variant or mutant form of one or several target proteins is carried out, wherein said variant or mutant forms are reintroduced into the cell or organism by an exogenous target nucleic acid as described above. The combination of knockout of an endogeneous gene and rescue by using mutated, e.g. partially deleted exogenous target has advantages compared to the use of a knockout cell. Further, this method is particularly suitable for identifying functional domains of the targeted protein. In a further preferred embodiment a comparison, e.g. of gene expression profiles and/or proteomes and/or phenotypic characteristics of at least two cells or organisms is carried out. These organisms are selected from: (i) a control cell or control organism without target gene inhibition, (ii) a cell or organism with target gene inhibition and (iii) a cell or organism with target gene inhibition plus target gene complementation by an exogenous target nucleic acid.

Furthermore, the RNA knockout complementation method may be used for is preparative purposes, e.g. for the affinity purification of proteins or protein complexes from eukaryotic cells, particularly mammalian cells and more particularly human cells. In this embodiment of the invention, the exogenous target nucleic acid preferably codes for a target protein which is fused to art affinity tag. This method is suitable for functional proteome analysis in mammalian cells, particularly human cells.

Another utility of the present invention could be a method of identifying gene function in an organism comprising the use of miRNA to inhibit the activity of a target gene of previously unknown function. Instead of the time consuming and laborious isolation of mutants by traditional genetic screening, functional genomics would envision determining the function of uncharacterized genes by employing the invention to reduce the amount and/or alter the timing of target gene activity. The invention could be used in determining potential targets for pharmaceutics, understanding normal and pathological events associated with development, determining signaling pathways responsible for postnatal development/aging, and the like.

The ease with which RNA can be introduced into an intact cell/organism containing the target gene allows the present invention to be used in high throughput screening (HTS). Solutions containing miRNAs that are capable of inhibiting the different expressed genes can be placed into individual wells positioned on a microtiter plate as an ordered array, and intact cells/organisms in each well can be assayed for any changes or modifications in behavior or development due to inhibition of target gene activity. The amplified RNA can be fed directly to, injected into, the cell/organism containing the target gene. Alternatively, the miRNA can be produced from a vector, as described herein. Vectors can be injected into, the cell/organism containing the target gene. The function of the target gene can be assayed from the effects it has on the cell/organism when gene activity is inhibited. This screening could be amenable to small subjects that can be processed in large number, for example: arabidopsis, bacteria, drosophila, fungi, nematodes, viruses, zebrafish, and tissue culture cells derived from mammals. A nematode or other organism that produces a calorimetric, fluorogenic, or luminescent signal in response to a regulated promoter (e.g., transfected with a reporter gene construct) can be assayed in an HTS format.

C. Screening Assays

miRNAs and their targets, as identified herein, are also suitable for use in methods to identify and/or characterize potential pharmacological agents, e.g. identifying new pharmacological agents from a collection of test substances and/or characterizing mechanisms of action and/or side effects of known pharmacological agents.

Thus, the present invention also relates to a system for identifying and/or characterizing pharmacological agents acting on at least one miRNA:target RNA pair comprising: (a) a eukaryotic cell or a eukaryotic non-human organism capable of expressing the target RNA, (b) at least one miRNA molecule capable of modulating (e.g., inhibiting) the expression of said target RNA, and (c) a test substance or a collection of test substances wherein pharmacological properties of said test substance or said collection are to be identified and/or characterized. Optionally, the system as described above can further comprise suitable controls.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J Med. Chem. 37:1233.

Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad Sci. 87:6378-6382); (Felici (1991) J. Mol. Biol. 222:301-310); (Ladner supra.)).

In a preferred embodiment, the library is a natural product library, e.g., a library produced by a bacterial, fungal, or yeast culture. In another preferred embodiment, the library is a synthetic compound library.

Compounds or agents identified according to such screening assays can be used therapeutically or prophylactically either alone or in combination, for example, with an miRNA of the invention, as described supra.

D. Differential Display

miRNAs and their targets, as identified herein, are also suitable for use in methods to identify and/or characterize important agents, e.g. potential causative agents, in a disease or disorder.

Thus, the present invention also relates to a system for identifying and/or characterizing at least one miRNA:target RNA pair as an important agent, e.g. a causative agent, for a disease or disorder comprising: (a) a eukaryotic normal cell capable of expressing the target RNA, (b) a eukaryotic diseased cell capable of expressing the target RNA, (c) a target RNA, and (d) at least one miRNA molecule capable of modulating (e.g., inhibiting) the expression of said target RNA, wherein the level or activity of the miRNA:target RNA in the normal cell and in the diseased cell are to be compared. Optionally, the system as described above can further comprise suitable controls. Changes in the relative level or activity of the miRNA:target RNA in the normal cell as compared to the relative level or activity of the miRNA:target RNA in the diseased cell are an indication that the miRNA:target RNA pair may be an important agent, e.g. a causative agent, for the disease or disorder.

The present invention also relates to a method of identifying an important agent, e.g. a causative agent, for a disease or disorder, comprising comparing an miRNA:target RNA level or activity in a normal cell to an miRNA:target RNA level or activity in a diseased cell, wherein an alteration in said level or activity is indicative of the agent being important or causative in a disease or disorder.

The level or activity of the miRNA:target RNAs in a normal or diseased cell can be measured and compared using any one of commonly known methods in the art. The level or activity of miRNA:target RNA can be measured and compared by first generating cDNA from the miRNA and the target RNA using DNA primers, and the level of miRNA:target RNA can then be determined using, e.g. quantitative PCR, which is well known in the art. Also within the scope of the present invention, the level of multiple miRNA:target RNA pairs in a normal cell as compared to a diseased cell can be determined by differential display analysis using DNA microarray technology. DNA microarray technologies are well known in the art. Microarrays can be analyzed by using, e.g., melting curve analysis technology, e.g., with SNP-Analysis Systems (Roche Applied Science). Melting curve analysis exploits the fact that melting temperatures of double-stranded polynucleotides are determined by G/C content, fragment length, and the degree of complementarity between strands, such that wild type and single nucleotide changes can be distinguished on the basis of differential melting curves.

This invention is further illustrated by the following examples which should not be construed as limiting. The contents of all references, patents and published patent applications cited throughout this application are incorporated herein by reference.

### EXAMPLES

Materials and Methods.

Total RNA isolation: RNA can be isolated by methods known in the art, taking care to maintain the presence of small miRNAs. For example, cells can be lysed in TRIzol reagent (Gibco-BRL), and total cellular RNA can be isolated according to the manufacturer's instructions. The presence of miRNA is confirmed, e.g., by separation on a denaturing 15% polyacrylamide gel, and the miRNA bands are excised and purified by routine methods. mRNA is isolated by routine methods.

Labeling miRNA: the isolated miRNA can be labeled by routine methods, including, but not limited to, radiolabeling, e.g., with ³²P, ³⁵S, etc.; fluorolabeling, e.g., with fluorescein or rhodamine; or labeling with some other detectable agent, e.g., digoxigenin or biotin. Additionally, miRNA can be labeled with a moiety useful for later purification of the miRNA/target RNA complex.

Formation of miRNA/target RNA complexes: miRNA and mRNA are combined and allowed to form complementary structures following routine procedures. As one example, not meant to be limiting, first isolated potential target mRNAs are denatured and renatured, e.g., denatured by a) adding 6 M urea or guanidine-HCl, or b) by heating to 100° C. for 3 min. Then, slow cooling allows the RNA to refold back to an active structure. Then, miRNA is added to the mRNA after renaturing. Alternatively, miRNA can also be added during renaturing (i.e., cooling).

Complexes are allowed to form by incubation in a complex formation mixture, e.g., PBS buffer containing 100 mM KCl or NaCl, or using a phenol/chloroform extraction (Elbashir et al., Genes Dev. 15:188-200 (2001)). Alternatively, complexes can be formed by the addition of isolated or synthetic miRNA to cell extracts or total RNA prepared by routine methods under appropriate conditions.

Isolation of miRNA/target RNA complex: Following complex formation, tcRNA transcription and/or PCR, a gel-shift type assay can be performed to detect and isolate miRNA/target RNA complexes using routine methods (see, e.g., Pyle et al., Proc. Natl. Acad. Sci. USA 87:8187-8191 (1990) for general methodology). In one example, the miRNA/target RNA mixture is run on a gel, e.g., a 5% polyacrylamide native gel comprising 50 mM Tris-OAc (pH 7.5) and 7.5, 10 or 12.5 mM Mg(OAc)2, side by side with mRNA alone, generally from the same mRNA isolation experiment. The formation of the miRNA/target RNA will cause a detectable shift in the apparent molecular weight of the complex. miRNA/target RNA complexes are then recovered from the gel. Alternatively, where labeled miRNA are used, miRNA/target RNA complexes can be separated on a gel or otherwise (e.g., on a column) and the label can then be used to detect and isolate the complexes using routine methods. Column purification of RNA can also be done using routine methods. As one example, RNA can be purified by ion exchange columns and/or FPLC (Amersham Biosciences). After RNA is bound to a column, various buffers including a salt gradient can be used to elute the bound RNA from the column.

In addition, where biotin-labeled miRNA are used, avidin-coated magnetic beads can be used to isolate miRNA/target RNA complexes. The beads may be any commercially available beads, e.g., IOBEADS-AVIDIN, superparamagnetic microspheres coated with avidin (Coulter); MTRAP Streptavidin beads (Active Motif); MAGACELL-Q beads (Cortex Biochem); DYNABEADS® (Dynal Biotech); or streptavidin magnetic particles (Roche Applied Science), used according to the manufacturer's instructions, or may be custom-synthesized.

It is important to note that the miRNA and target RNA in a complex need not be totally complementary; interaction between miRNA and mRNA that are more complementary, e.g., more than 80% complementary, e.g., 81%, 82%, 83%, 84%, 85%, 86%, 87%, 88%, 89%, 90%, 91%, 92%, 93%, 94%, 95%, 96%, 97%, 98%, 99%, or 100% complementary, are considered to be stable, as opposed to those interactions characterized by less complementarity. When areas of non-complementarity exist, stability is greater when the non-complementarity occurs primarily in the center of the sequence of the miRNA as opposed to at or near the ends. Where the degree of complementarity is in doubt, crosslinking reagents, including those reagents described herein, e.g., Compound 1, may be used to stabilize the miRNA/target RNA complex. tcRNA Transcription Reactions: Transcription can be achieved by routine methods, including by RNA dependent RNA polymerase (RdRP) transcription reactions or by reverse transcription using routine protocols. As one example, recombinant RdRP can be used. (see, for example, Oh et al., J. Virology, 73(9):7694-7702 (1999)). Briefly, the reaction is carried out in a total volume of 25 μl containing 50 mM Tris-HCl (pH 8.0); 50 mM NaCl; 5 mM MgCl₂; 100 mM potassium glutamate; 1 mM DTT; 10% glycerol; 20 μg of actinomycin D per ml (Sigma); 20 U of RNase inhibitor (Promega); 0.5 mM each ATP, CTP, GTP, and UTP (for radiolabeled product, any labeled nucleotide, e.g., 10 μCi of [³²P]UTP (3,000 Ci/mmol; NEN Research Products) can be used); about 0.1-5 μg of purified template mRNA; about 200 ng of purified RdRP, and about 10-20 pmol of miRNA. The reaction mixture is incubated at 25° C. for 2 h unless otherwise indicated. After the RdRP reaction, 35 μl of double-distilled H₂O containing 20 μg of glycogen (Boehringer Mannheim) and 60 μL of acidic phenol emulsion (phenol-chloroform [Ambion]-10% SDS-0.5 M EDTA [1:1:0.2:0.04]) are added to the reaction mixture to terminate the reactions. The RNAs are then precipitated with 2.5 volumes of 5 M ammonium acetate-isopropanol (1:5), followed by washing with 70% ethanol.

Fill-In Reaction: As is shown in FIG. 1C, in some embodiments of the invention, following transcription of the tcRNA strand downstream (in the 3′ direction) of the miRNA, routine methods can be used to fill in the sequence in the other direction (the 5′ direction), e.g., incubation with Taq polymerase under appropriate conditions, e.g., as described in Wang et al., Biochemistry 40:6458-6464 (2001). This may be used in conjunction with any of the embodiments of the present invention, and is not limited to the method as shown in FIG. 1C.

Ligation of Adaptors to the 5′ or 3′ end: 3′ adaptors can be ligated to the ends of the tcRNA/target RNA complex using routine methods. As one example, first the complex is dephosphorylated. Next, 3′ adaptors are ligated to dephosphorylated complexes using T4 DNA ligase (Amersham-Pharmacia). The reaction is stopped, the products recovered, and the complex is then 5′ phosphorylated, e.g., using T4 polynucleotide kinase (NEB). Again, the reaction is stopped and the products recovered. Finally, the 5′ adaptors are ligated on. See, e.g., Elbashir et al., (2001) supra; Moore et al., Science 256:992-997 (1992). The adaptors can be of any sequence and need not be complementary to the target RNA. In some embodiments, the adaptors will contain restriction enzyme recognition sites to facilitate cloning.

PCR amplification of Transcription Products: Routine Polymerase Chain Reaction (PCR) techniques can be used to amplify the transcription products for cloning or direct sequencing. PCR primers may be designed to be complementary to linkers of known sequence added to the 3′ or 5′ ends of the transcription products as described herein, or to one linker and the miRNA, if the sequence of the miRNA is known, as is shown in FIGS. 1B and 2B. PCR is carried out by routine methods, e.g., as described in Lee et al., EMBO J. 21(17):4663-4670 (2002); Lagos-Quintana et al., Science 294:853-858 (2001).

PAGE Separation of PCR or Transcription Products: The PCR or transcription products are resuspended in a non-denaturing loading buffer containing xylene cyanol and bromophenol blue. The products are resolved on a non-denaturing gel. The gels are stained with ethidium bromide, photographed to locate the template positions, and then dried after fixing. The dried gels are exposed to X-ray film for autoradiography. The products can be recovered from the gel by standard methods.

Agarose Gel Separation of PCR or Transcription Products: After resuspension in an appropriate loading buffer, the products are resolved on a standard low-melt agarose gel. The gels are stained with ethidium bromide, photographed to locate the template positions, and then dried after fixing. The dried gels are exposed to X-ray film for autoradiography. The products can be recovered from the gel by routine methods; e.g., melting and phenol extraction.

UV Crosslinking: The crosslinking compounds of the present invention may be used following routine methods, such as those described in Chiu and Rana, Molecular Cell 10:549-561 (2002) and Wang et al., J. Biol. Chem. 271(29):16995-16998 (1996). For in vivo crosslinking, cells are exposed to long wave UV (340-380 nm) for 30 sec to 15 min. If heating occurs, plates are incubated on ice or in a circulation bath that can maintain the desired temperature.

### Example 1

### Identification of miRNAs and their Targets when Interactions are Stable, and the Sequence of the miRNA does not need to be Known

As is illustrated in FIG. 1A, following formation of the miRNA/target RNA complex as described herein, RdRP or RT is used to synthesize full-length tcRNA using the miRNA as a primer to initiate transcription in the 3′ direction from the miRNA to form a complete complex of tcRNA/target RNA. Next, adapters of known sequences are ligated to one or more ends of the tcRNA/target RNA complex using DNA or RNA ligase enzymes, e.g., T4 DNA ligase, and standard procedures. Then, using PCR primers complementary to the adapters, DNA is synthesized, which can then be cloned and/or directly sequenced. Where the sequence of the miRNA is known, one of the PCR primers can be homologous to the miRNA, as is shown in FIG. 1B. In one alternative method, as shown in FIG. 1C, the tcRNA strand is extended from the 5′ end of the miRNA using standard methods prior to addition of the adaptors.

### Example 2

### Identification of miRNAs and their Targets when Interactions are Dynamic and not Stable for RNA Isolation, and the Sequence of the miRNA does not need to be Known

As illustrated in FIG. 2A, the bifunctional Compound 1 (bifunctional biotin-aminopentyl 8-hydroxypsoralen; FIG. 6A) can be added to the cells or cell extracts and exposed to long wave UV (360 nm) to freeze RNA-RNA interactions. Alternatively, the compound can be added to the miRNA/target RNA complex formation mixture and then exposed to long wave UV (360 nm). Crosslinked miRNA/target RNA complexes can easily be immobilized on avidin-coated magnetic beads and further reactions can be carried out on beads or solution phase. RdRP or RT is used to synthesize full length tcRNA using the target RNA as a template, with the miRNA serving as a primer. Adapters of known sequences are then ligated to one or more of the ends of the tcRNA/target RNA complex using DNA or RNA ligase enzymes, e.g., T4 DNA ligase, and standard procedures. Then, using PCR primers complementary to the adapters, DNA can be synthesized. The PCR primers can be designed to incorporate a restriction enzyme recognition site.

As shown in FIG. 2B, where the miRNA is known, one of the PCR primers can be homologous to the miRNA.

### Example 3

### Identification of miRNAs and their Targets when the miRNA Sequence is Known and their Interactions are Stable

As illustrated in FIG. 3, synthetic miRNAs are made incorporating amino-modified C or U in the miRNA sequence for further labeling with biotin or psoralen biotin using either Compound 2 (FIG. 6B) or Compound 3 (FIG. 6C). This labeled miRNA is added to cells or cell extracts prepared by routine methods, miRNA/target RNA complexes are allowed to form, and total RNA is isolated. The biotin label is then used to immobilize the complexes on avidin-coated magnetic beads following the manufacturer's instructions. RdRP or RT is then used to synthesize full length tcRNA using target RNA as a template and the miRNA as a primer. Adapters of known sequences are ligated to the ends of the tcRNA/target RNA complex using DNA or RNA ligase enzymes, e.g., T4 DNA ligase, using standard methods. Then, using PCR primers complementary to the adapters, DNA is synthesized. The PCR primers can be designed to have restriction enzyme recognition sequences to allow for convenient cloning. Although it is not shown on FIG. 3, one PCR primer may be homologous to the miRNA. Additionally, the tcRNA strand may be extended 5′ from the miRNA by standard methods prior to the addition of the adaptors.

### Example 4

### Identification of miRNAs and their Targets when the miRNA Sequence is Known and their Interactions are Dynamic and not Stable for RNA Isolation

As illustrated in FIG. 4, Compound 4, an activated ester of hexanoic acid linked with a biotin and a 4-thioUracil, (FIG. 7A) or Compound 5, an activated ester of hexanoic acid linked with a biotin and an 8-hydroxy-psoralen (FIG. 7B) is used to label amino-U or amino-C in a synthetic miRNA and is added to cells or cell extracts prepared by routine methods, or the complex formation mixture. miRNA/target RNA complexes are allowed to form, and the mixture is then exposed to long wave UV (360 nm) to freeze RNA-RNA interactions as described above. Crosslinked miRNA/target RNA complexes are immobilized on avidin-coated magnetic beads and further reactions can then be carried out on beads or in solution phase. RdRP or RT are used to synthesize full length tcRNA. Adapters of known sequences are then ligated to the ends of the complexes using DNA or RNA ligases, e.g., T4 DNA ligase, using routine methods. Then, using PCR primers complementary to the adapters or homologous to the miRNA, DNA can be synthesized. The PCR primers can be designed to have restriction enzyme recognition sequences to allow for convenient cloning. Although not shown on FIG. 4, one PCR primer may be homologous to the miRNA. Additionally, the tcRNA strand may be extended 5′ from the miRNA by standard methods prior to the addition of the adaptors.

### Example 5

### Identification of Target RNAs In Vivo

miRNA, either synthetic or natural, can be labeled with biotin and delivered to a cell or cells. The methods as described above can be used to identify in vivo targets. Compound 1 may be added to stabilize any dynamic miRNA/target RNA interaction by photocrosslinking. In addition, psoralen-biotin and 4-thioU-biotin labeled miRNA can be added to the cells, photocrosslinked in vivo, thus identifying in vivo targets.

### Example 6

### Identification of miRNAs and their Targets Using Strategies in which the RT Step Involves a DNA Primer

It has recently been observed that reverse transcription is more inefficient when priming comes from a DNA primer as compared to an RNA primer (e.g., an miRNA primer) as described in Examples 1-5. Consequently, strategies were developed in which the RT step involve a DNA primer rather than relying on the miRNA to primer synthesis of the strand complementary to the target RNA. (See FIG. 8A).

Briefly, biotinylated miRNA is generated and transfected into a cell. miRNA-target RNA complexes are allowed to form. In order to increase complex stability, psoralen or psoralen derivatives and long wave UV light are used to cross-link the miRNA-target complex (FIG. 8B). Total mRNA is isolated from the cell and streptavidin coated beads added to the extract. The beads bind to the biotin tag on the end of the miRNA and provide a convenient means to enrich for the miRNA-target complexes.

In an optional step, the enrichment can be monitored by using, PCP, e.g. α-³²P-cordycepin 5′ triphosphate (e.g. 5000 Ci/mmol; New England Nuclear) or cytidine-3′,5′-bis (phosphate) [5′-³²P], to label the 3′ end of the target mRNA, and radiolabeled RNA complexes can be separated by polyacrylamide gel electrophoresis. The detection of multiple radiolabeled bands, e.g. greater than 2, 3, 4 or 5 radiolabeled bands, can indicate the presence of contaminating RNA, e.g. non-target RNAs. The entire procedure can then be repeated and optimized for efficiency until one predominant band representing an miRNA-target complex is visible on the gel. Parameters that can be varied to optimize efficiency of the procedure include, but are not limited to, salt concentration, presence of non-denaturing detergents, e.g. NP-40 and sarkosyl, and the temperature at which the complex is formed.

Treating the isolated complex with photo-reversible UV 254 generates unmodified (i.e., uncross-linked) RNA species. The complementary DNA form of the miRNA can be added as a primer for subsequent synthesis (RT) reactions. Once the primer is added, a complementary DNA of the target RNA can be produced by using standard procedures. Finally, standard amplification techniques and sequencing reactions can be performed in order to determine the sequence of the target.

Additional strategies were developed in which the miRNA is modified as follows. In a first strategy, the miRNA has an amino-modified nucleotide, e.g. uridine or cytidine, in order to target crosslinking by psoralen or other crosslinkers known in the art to the amino-modified nucleotide within the miRNA (FIG. 8C). In a second strategy, the miRNA has an amino-modified nucleotide at the 3′ end of the miRNA, in order to target crosslinking by psoralen or other crosslinkers known in the art to the amino-modified 3′ nucleotide within the miRNA (FIG. 8E). In a third strategy, the miRNA has an amino-modified nucleotide at the 5′end of the miRNA, in order to target crosslinking by psoralen or other crosslinkers known in the art to the amino-modified 5′ nucleotide within the miRNA (FIG. 8F). In these three additional strategies, the miRNA is further labeled with any one of psoralen, psoralen derivatives or other crosslinkers known in the art. In a fourth additional strategy in which the miRNA is modified, the miRNA has a photoactive nucleotide, e.g. 4-thio uridine or 4-thio thymidine or a 6-thio guanosine, in order to target crosslinking to the photoactive nucleoside within the miRNA (FIG. 8D). In each additional strategy, long wave UV light is used to cross-link the miRNA-target complex. Total mRNA is isolated from the cell and streptavidin coated beads and the protocol proceeds as described above.

A modification of the approach described above allows for the identification of miRNAs and their targets even if the sequence of the miRNA is unknown. In such an instance, a psoralen-biotin conjugate with a linker is added to cells and binds to RNA within the cells. Crosslinked products are isolated using streptavidin beads and the protocol proceeds basically as described above, except that a poly A primer is used in the RT reactions. An adapter primer is used for the PCR step needed to amplify the target prior to sequencing.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed:

1. A method for identifying an miRNA and its target RNA, the method comprising
(a) obtaining an miRNA/target RNA complex;
(b) optionally crosslinking the complex;
(c) transcribing target complementary RNA (tcRNA) from the target RNA;
(d) synthesizing cDNA complementary to the tcRNA; and
(e) sequencing the cDNA,
thereby identifying the miRNA and its target.

2. The method of claim 1, wherein obtaining the miRNA/target RNA complex comprises
(a) obtaining miRNA;
(b) administering miRNA to a cell or cell extract; and
(c) allowing miRNA/target RNA complexes to form;
thereby obtaining an miRNA/target RNA complex.

3. The method of claim 1, further comprising:
(a) contacting the miRNA/target RNA complex with a bifunctional biotin-aminopentyl 8-hydroxypsoralen (Compound 1); and
(b) photocrosslinking the complex.

4. The method of claim 3, further comprising immobilizing the miRNA/target RNA complex using avidin-coated magnetic beads.

5. A method for identifying a target RNA of an miRNA, the method comprising
(a) obtaining a modified miRNA comprising an amino-modified cytosine or amino-modified uracil;
(b) contacting the miRNA with a target RNA;
(c) allowing an miRNA/target RNA complex to form;
(d) labeling the complex with a biotin compound selected from an NHS activated ester of biotin butanoic acid (Compound 2) or photocleavable biotin (Compound 3);
(e) transcribing target complementary RNA (tcRNA) from the target RNA;
(f) synthesizing cDNA complementary to the tcRNA; and
(g) sequencing the cDNA,
thereby identifying the target RNA.

6. The method of claim 5, further comprising immobilizing the miRNA/target RNA complex using avidin-coated magnetic beads.

7. A method for identifying the target RNA of an miRNA, the method comprising
(a) obtaining an miRNA having a known sequence;
(b) contacting the miRNA with a target RNA;
(c) allowing an miRNA/target RNA complex to form;
(d) labeling the miRNA/target RNA complex with a compound selected from an activated ester of hexanoic acid linked with a biotin and a 4-thio-uracil (Compound 4) or an activated ester of hexanoic acid linked with a biotin and 8-hydroxy-psoralen (Compound 5);
(e) optionally crosslinking the miRNA/target RNA complex;
(f) transcribing target complementary RNA (tcRNA) from the target RNA;
(g) synthesizing cDNA complementary to the tcRNA; and
(h) sequencing the cDNA,
thereby identifying the target RNA.

8. The method of claim 7, further comprising the step of immobilizing the complex on avidin-coated magnetic beads.

9. The method of any one of claims 1-8 wherein the miRNA/target RNA complex forms in a cell-free solution.

10. The method of any one of claims 1-8 wherein the miRNA/target RNA complex forms in a cell.

11. A method for identifying the target RNA of an miRNA, the method comprising
(a) contacting a biotin-labeled miRNA having a known sequence with a target RNA;
(b) allowing an miRNA/target RNA complex to form;
(c) crosslinking the miRNA/target RNA complex, e.g. with a psoralen compound or other crosslinking agent;
(d) immobilizing the complex on avidin-coated beads;
(e) reversing the crosslink;
(f) transcribing a complementary strand from the target RNA using reverse transcriptase and a cDNA primer, e.g., a primer having a sequence corresponding to the miRNA;
(g) synthesizing cDNA complementary to the transcribed strand of (f); and
(h) sequencing the cDNA,
thereby identifying the target RNA.

12. The method of claim 11, wherein step (c) comprises crosslinking the miRNA/target RNA complex via a modified nucleotide in the miRNA.

13. The method of claim 12, wherein the nucleotide is a uridine or cytidine within the miRNA.

14. The method of claim 13, wherein the modified nucleotide is an amino-modified uridine or amino-modified cytidine within the miRNA.

15. The method of claim 12, wherein the nucleotide is a uridine, thymidine or guanosine within the miRNA.

16. The method of claim 15, wherein the modified nucleotide is a 4-thio uridine, 4-thio thymidine or 6-thio guanosine within the miRNA.

17. The method of claim 12, wherein the crosslink is targeted to the 5′ end of the miRNA.

18. The method of claim 17, wherein the crosslink comprises an amino-modified 5′nucleotide.

19. The method of claim 18, wherein the crosslink comprises an amino-modified 5′ uridine or cytidine.

20. The method of claim 12, wherein the crosslink is targeted to the 3′ end of the miRNA.

21. The method of claim 20, wherein the crosslink comprises an amino-modified 3′ nucleotide.

22. The method of claim 21, wherein the crosslink comprises an amino-modified 3′ uridine or cytidine.

23. The method of claim 11 wherein the miRNA/target RNA complex forms in a cell.

24. The method of claim 11 wherein the miRNA/target RNA complex forms in a cell-free solution.

25. A method for identifying a target RNA of an miRNA, the method comprising
(a) contacting a cell with a psoralen-biotin conjugate such that the conjugate binds to target RNA within the cell;
(b) allowing the target RNA to form a complex with miRNA within the cell;
(c) immobilizing the miRNA:target RNA complex on avidin-coated beads;
(d) reversing the crosslink;
(e) transcribing a complementary strand from the target RNA using reverse transcriptase and a poly A primer;
(f) synthesizing cDNA complementary to the transcribed strand of (f); and
(g) sequencing the cDNA,
thereby identifying the target RNA.

26. A method for modulating the expression of a target RNA in a cell, the method comprising
(a) identifying an miRNA that affects the expression of the target RNA using the method of any one of claims 1, 5, 7, 11 or 25; and
(b) modulating the activity of the miRNA in the cell
thereby modulating the expression of the target.

27. The method of claim 26 wherein the expression of the target RNA is increased or decreased.

28. The method of claim 26 wherein the target RNA encodes a gene involved in a proliferative or differentiative disease.
